(19) United States
(12) Patent Application Publication
(10) Pub. No.: US 2013/0280755 A1
(43) Pub. Date: Oct. 24, 2013

(54) THERAPEUTIC METHODS FOR SOLID DELIVERY

(71) Applicant: Christopher Hubert, Shoreline, WA (US)

(72) Inventor: Christopher Hubert, Shoreline, WA (US)

(73) Assignee: Fred Hutchinson Cancer Research Center, Seattle, WA (US)

(21) Appl. No.: 13/988,309

(22) PCT Filed: Nov. 23, 2011
(86) PCT No.: PCT/US11/62046

§ 371 Date: Jul. 8, 2013

Related U.S. Application Data
(60) Provisional application No. 61/416,699, filed on Nov. 23, 2010.

(51) Int. Cl. G01N 33/50; A61M 5/32

(52) U.S. Cl. CPC G01N 33/5011; A61M 5/3295
USPC 435/32; 604/173; 604/506

(57) ABSTRACT

A fluid-delivery device includes an array of needles. The needle can deposit a hollow and/or porous tube into a tissue of a subject, and the porous tube can contain one or more fluid agents. The hollow and/or porous tube can control the rate at which the agents diffuse into the tissue. The device can simultaneously deliver a plurality of porous tubes along parallel axes in a tissue in vivo. If thereafter resected, the tissue can be sectioned for evaluation of an effect of each agent on the tissue; based on the evaluation, candidate agents or subjects can be selected or deselected for clinical trials or therapy.

### RELATED APPLICATION INFORMATION

This application claims the benefit of priority under 35 U.S.C. section 119(e) to U.S. Provisional Application 61/416,699, filed Nov. 23, 2010, the contents of which are incorporated by reference in its entirety.

### BACKGROUND

Numerous cancer-related agents are under preclinical and clinical trials and evaluations at any particular time; however, most of them will fail to advance. In fact, it is estimated that more than 90% of cancer-related therapeutics will fail phase I or II clinical trial evaluation. The failure rate in phase III trials is almost 50%, and the cost of new drug development from discovery through phase III trials is between $0.8 billion and $1.7 billion and can take between eight and ten years.

In addition, many subjects fail to respond even to standard drugs that have been shown to be efficacious. For reasons that are not currently well understood or easily evaluated, some individual subjects do not respond to standard drug therapy. One significant challenge in the field of oncology is to exclude drug selection for individual subjects having cell autonomous resistance to a candidate drug to reduce the risk of unnecessary side effects without concomitant benefit. A related problem is that excessive systemic concentrations are required for many oncology drug candidates in efforts to achieve a desired concentration at a tumor site, an issue compounded by poor drug penetration in many under-vascularized tumors (Tunggal et al., 1999 Clin. Canc. Res. 5:1583).

Clearly there is a need in the art for improved devices and methods for testing cancer therapies, including improved methodologies for performing efficient pre-clinical and clinical studies of candidate oncology medicines, and for identifying therapeutics having increased likelihood of benefitting individual subjects. The present invention addresses these and similar needs, and offers other related advantages.

### SUMMARY OF THE INVENTION

In some embodiments, the disclosure involves a device for constrained solid delivery of one or more fluid agents to a tissue, comprising one or more needles, each configured to receive a hollow and/or porous tube; one or more hollow and/or porous tubes, each configured to contain at least one fluid agent. In some aspects, the device further comprises an actuator configured to push a tube from a needle upon activation. The actuator may be a plunger or a pump. In some aspects, the device comprises from about 1 to 1,000 needles. In some aspects, the device comprises from 1 to about 500 tubes.

According to some aspects of the invention, the device further comprises one or more reservoirs each in communication with a respective one of said one or more needle. In some embodiments, at least one of said reservoirs contains a hollow and/or porous tube. The device may comprise 2, 3, 4, 5, 6, 7, 8, 9, 10, 15, 20, 100, or more reservoirs.

In some embodiments, each of the one or more needles is not permeable to the one or more fluid agents. In a further embodiment, the needles are not porous needles or have no pores along the length of the needle. In some embodiments, at least one of said one or more needles can deliver two or more porous tubes to a specific location within the tissue.

In some embodiments, the porous tube comprises a plurality of pores. In some of these cases, the fluid agents may be capable of diffusing through the pores at a diffusion rate when the porous tube is embedded in animal tissue, wherein the diffusion rate may be controlled by the pore size. In some aspects, the pore size may be within a range between about 1 nm and 5 micrometers. In some aspects, the pore diameter may be less than about 1 nm, 2 nm, 5 nm, 10 nm, 20 nm, 50 nm, 100 nm, 200 nm, 500 nm, 1 micrometer, or 5 micrometers.

In some embodiments, the hollow and/or porous tube is biocompatible, permeable, and/or scissile. In some embodiments, at least one of the hollow and/or porous tubes is essentially insoluble in organic solvents, water, or a combination thereof. In some embodiments, at least one of the hollow and/or porous tubes is essentially inert to acid. The tubes can comprise polysulfone, polyamine, polyamide, polycarbonate, polycarbamate, polyurethane, polyester, polyether, polyolefin, polyaromatic, a cross-linked polymer, polylactic acid, or a combination or co-polymer of any of the foregoing. In some aspects, the hollow and/or porous tubes comprise polysulfone. In some cases, each of said one or more hollow and/or porous tubes comprises a hydrogel.

The hollow and/or porous tubes may comprise the same fluid agent or different fluid agents. In some embodiment, none of the hollow and/or porous tubes comprises the same fluid agent as the agent in any other tubes. In other embodiments, at least one porous tube comprises two or more fluid agents. In certain other embodiments, at least two of the porous tubes comprise a same fluid agent. In a further embodiment, the concentrations of the same fluid agent in different porous tubes are different. In some embodiments, at least one of said hollow and/or porous tubes comprises at least one indicator particle. The indicator particle is selected from the group consisting of a metallic particle, a fluorescent dye, a quantum dot, a quantum barcode, a radiographic contrast agent, and a magnetic resonance imaging contrast agent. In some preferred embodiments, the indicator particle is a dye.

In some embodiments, the tissue may be an animal tissue or a human tissue. In a preferred embodiment, the tissue is a tumor. The tumor may be a benign tumor or a malignant tumor. In some aspect, the tumor comprises at least one cancer cell selected from the group consisting of a leukemia cell, a pancreatic cancer cell, a prostate cancer cell, a breast cancer cell, a colon cancer cell, a lung cancer cell, a brain cancer cell, a glioma cancer cell, a melanoma cell, a renal cancer cell, and an ovarian cancer cell. In some other aspects, the tissue is selected from the group consisting of brain, liver, lung, kidney, prostate, ovary, spleen, lymph node, thyroid, pancreas, heart, muscle, intestine, larynx, esophagus, stomach, nerve, brain, thymus, testis, skin, bone, breast, uterus, and bladder.

In some embodiments, the fluid agents may comprise an anti-cancer agent, an anti-inflammatory agent, an anti-infective agent, a regenerative agent, a relaxing agent, an apoptosis-inhibiting agent, an apoptosis-inducing agent, an anti-coagulatory agent, a dermatological agent, a growth-stimulating agent, a vasodilating agent, a vasorestricting agent, a analgesic agent, or an anti-allergic agent. In some embodiments, the fluid agents may comprise a protein, a peptide, a polypeptide, a peptidomimetic, an antibody, a small molecule, a small interfering RNA-encoding polynucleotide, an antisense RNA-encoding polynucleotide, or a ribozyme-encoding polynucleotide. In some embodiments, the fluid agents comprise an anti-cancer agent. In a further aspect, the anti-cancer agent is a small molecule agent. In a further aspect, the small molecule has molecular weight of less than 10³ Dalton.

The drug-delivery device may comprise two or more needles. In some embodiments, none of the needles comprises the same fluid agent as the agent in any other needles. In other embodiments, at least one of the needles comprises two or more porous tubes. In yet other embodiments, at least one of the needles comprises two or more fluid agents. In yet other embodiments, at least two of the needles comprise a same fluid agent. In a further embodiment, the concentrations of the same fluid agent in different needles are different.

Some aspects of the present disclosure pertain to a method for spatially restricted solid delivery of one or more fluid agents to a tissue of an organism, comprising the steps of loading one or more hollow and/or porous tubes with a fluid agent; inserting said one or more hollow and/or porous tubes into a tissue using one or more needles; and delivering the content of said one or more hollow and/or porous tubes to said tissue at least partially by diffusion through pores of said one or more hollow and/or porous tubes. In some cases, the fluid agent is delivered solely by diffusion. In some cases, each of said one or more hollow and/or porous tubes comprises a hydrogel. In some embodiments, each of the one or more needles is not permeable to the one or more fluid agent. In a further embodiment, the needles are not porous needle or have no pores along its length.

In some aspects of the invention, the hollow and/or porous tubes comprise polysulfone, polyamine, polyamide, polycarbonate, polycarbamate, polyurethane, polyester, polyether, polyolefin, polyaromatic, polylactic acid, a cross-linked polymer, or a combination or co-polymer of any of the foregoing. In some aspects, the average pore diameter is within a range of about 1 nm to about 5 micrometers. In some aspects, the pore diameter is less than about 1 nm, 2 nm, 5 nm, 10 nm, 20 nm, 50 nm, 100 nm, 200 nm, 500 nm, 1 micrometer, or 5 micrometers.

In some embodiments, each of the one or more needles is not permeable to the one or more fluid agents. In a further embodiment, the needles are not porous needles or have no pores along the length of the needle. In some embodiments, at least one of said one or more needles can deliver two or more porous tubes to a specific location within the tissue.

According to certain embodiments, two or more hollow and/or porous tubes may be inserted. The hollow and/or porous tubes may comprise the same fluid agent or different fluid agents. In some embodiment, none of the hollow and/or porous tubes comprises the same fluid agent as the agent in any other tubes. In other embodiments, at least two of the porous tubes comprise the same fluid agent. In a further embodiment, the concentrations of the same agent in different porous tubes are different. In other embodiments, at least one porous tube comprises two or more fluid agents. In some embodiments, the fluid agents comprise a gene therapy agent; a chemotherapy agent; a small molecule; an antibody; a protein; a vector expressing a cDNA or shRNA; a small interfering RNA; an antisense RNA; a ribozyme; a detectable label; a therapeutic protein, a polypeptide, or a peptidomimetic; or a microRNA. In other embodiments, the fluid agents comprise a protein, a peptide, a polypeptide, a peptidomimetic, an antibodie, a small molecule, a small interfering RNA-encoding polynucleotide, an antisense RNA-encoding polynucleotide, or a ribozyme-encoding polynucleotide. In some embodiments, the fluid agents comprise an anti-cancer agent. In a further aspect, the anti-cancer agent is a small molecule agent. In a further aspect, the small molecule has molecular weight of less than 10³ Dalton.

In some embodiments, the tissue comprises a tumor, which may be a benign tumor or a malignant tumor. Additionally, the tumor may be a primary tumor, an invasive tumor or a metastatic tumor. The tumor may comprise a prostate cancer cell, a breast cancer cell, a colon cancer cell, a lung cancer cell, a brain cancer cell, and an ovarian cancer cell. The tumor may also comprise adenoma, adenocarcinoma, squamous cell carcinoma, basal cell carcinoma, small cell carcinoma, large cell undifferentiated carcinoma, chondrosarcoma or fibrosarcoma. The solid tissue may be selected from brain, liver, lung, kidney, prostate, ovary, spleen, lymph node, thyroid, pancreas, heart, muscle, intestine, larynx, esophagus, stomach, nerve, brain, thymus, testis, skin, bone, breast, uterus, or bladder.

Also provided herein according to certain embodiments is a method of evaluating the effect of one or more fluid agents on a tissue of an organism, comprising the steps of: i) inserting one or more hollow and/or porous tubes into a tissue using one or more needle; ii) delivering the content of the hollow and/or porous tube to the tissue at least partially by diffusion through pores of the hollow and/or porous tube; and iii) evaluating the effects of the fluid agents on the tissue. In some embodiments the effect of one or more fluid agents on a tissue of an organism may be evaluated in a method in which the one or more fluid agents have been pre-delivered to the tissue by a method comprising the steps of: i) inserting one or more hollow and/or porous tubes into a tissue using one or more needle; ii) delivering the content of the hollow and/or porous tube to the tissue at least partially by diffusion through pores of the hollow and/or porous tube.

In some embodiments, the content is delivered solely by diffusion. In some embodiment, the hollow and/or porous tubes comprise polysulfone, polyamine, polyamide, polycarbonate, polycarbamate, polyurethane, polyester, polyether, polyolefin, polylactic acid, polyaromatic, a cross-linked polymer, or a combination or co-polymer of any of the foregoing. In some aspects of the invention, the diffusion rates of the fluid agents are controlled by the pore size. The average pore diameter may be within a range of about 1 nm to about 5 micrometers. In some aspects, the pore diameter is less than about 1 nm, 2 nm, 5 nm, 10 nm, 20 nm, 50 nm, 100 nm, 200 nm, 500 nm, 1 micrometer, or 5 micrometers.

In some embodiments, the fluid agents may comprise a gene therapy agent; a chemotherapy agent; a small molecule; an antibody; a protein; a small interfering RNA; an antisense RNA; a ribozyme; a detectable label; a therapeutic protein, a polypeptide, or a peptidomimetic; or a microRNA. In other embodiments, the fluid agents comprise a protein, a peptide, a polypeptide, a peptidomimetic, an antibody, a small molecule, a small interfering RNA-encoding polynucleotide, an antisense RNA-encoding polynucleotide, or a ribozyme-encoding polynucleotide. In some embodiments, the fluid agents comprise an anti-cancer agent. In a further aspect, the anti-cancer agent is a small molecule agent. In a further aspect, the small molecule has molecular weight of less than 10³ Dalton.

According to certain embodiments, two or more porous tubes may be inserted. In some embodiments, each porous tube may contain a same or a different fluid agent. In certain other embodiments, at least one porous tube contains at least two fluid agents. In some embodiments, the porous tube may be inserted along parallel axes of the tissue. The delivery of fluid agents is restricted to the tissue, such that the agents are delivered to the tissue at or below systematically detectable concentration.

In some embodiments, the insertion occurs with an actuator. The actuator may be a plunger or a pump. In some embodiment, the hollow and/or porous tubes are inserted along parallel axes of the tissue. After the insertion, the hollow and/or porous tubes may stay in the tissue for a selected period of time. The selected period of time may be at least one minute.

In some embodiments, the tissue comprises a tumor, which may be a benign tumor or a malignant tumor. Additionally, the tumor may be a primary tumor, an invasive tumor and a metastatic tumor. The tumor may comprise a prostate cancer cell, a breast cancer cell, a colon cancer cell, a lung cancer cell, a brain cancer cell, and an ovarian cancer cell. The tumor may also comprise adenoma, adenocarcinoma, squamous cell carcinoma, basal cell carcinoma, small cell carcinoma, large cell undifferentiated carcinoma, chondrosarcoma or fibrosarcoma. The solid tissue may be selected from brain, liver, lung, kidney, prostate, ovary, spleen, lymph node, thyroid, pancreas, heart, muscle, intestine, larynx, esophagus, stomach, nerve, brain, thymus, testis, skin, bone, breast, uterus, or bladder.

In some embodiment, the evaluation comprises excising at least one portion of the tissue after introducing the fluid agents. In some embodiments, the evaluation is carried out on at least one portion of the tissue that has previously been excised. In some aspects, the excising occurs at a selected period of time after introducing the fluid agents. In some embodiments, the selected period of time is a range between about 1 minute and 96 hours. In certain embodiments, the selected period of time is a period exceeding one week. In some embodiments, the selected period of time is between one week and six months. The evaluation can be based, for example, detectable indicator compounds, nanoparticles, nanostructures or other compositions that comprise a reporter molecule which provides a detectable signal indicating the physiological status of a cell, such as a vital dye (e.g., Trypan blue), a colorimetric pH indicator, a fluorescent compound that can exhibit distinct fluorescence as a function of any of a number of cellular physiological parameters (e.g., pH, intracellular Ca²⁺ or other physiologically relevant ion concentration, mitochondrial membrane potential, plasma membrane potential, etc.). In some embodiments, at least one hollow and/or porous tube comprise at least one indicator compound. In some other embodiments, the evaluation comprises imaging the solid tissue. The imaging may be radiographic imaging, magnetic resonance imaging, positron emission tomogoraphy, or biophotonic imaging. The imaging may occur before, during, or after introduction of said candidate agents. In some embodiments, the evaluation comprises detecting an altered physiological state. In some embodiments, the evaluation comprises determining and comparing the effects of at least two of the fluid agents on adjacent positions within the region of the solid tissue. In some embodiments, the evaluation comprises determining the effects of at least two of the fluid agents on a same position within the region of the solid tissue. The evaluation obtained in various embodiments may be used for selecting a therapeutic agent for clinical trial.

According to certain other embodiments, there is provided a method of rating a candidate agent for development into a therapeutic agent, comprising the steps of: (i) inserting one or more porous tubes containing one or more candidate agents into a tissue using one or more needles; (ii) delivering the content of said one or more porous tubes into the tissue at least partially by diffusion through pores of said one or more porous tubes; and iii) evaluating the effect of said one or more candidate agents on the tissue. In certain further embodiments, the method comprises one of (i) selecting at least one of said agents based on said evaluation; (ii) deselecting at least one of said agents based on said evaluation; and (iii) prioritizing at least two of said agents based on said evaluating. In certain embodiments, said inserting is performed with a needle array device. In certain other embodiments, said one or more candidate agents are delivered at or below systematically detectable concentration. In certain other embodiments, said tissue is a tumor. In certain other embodiments, said one or more candidate agents comprise at least one position marker. In certain other embodiments, said one or more candidate agents comprise at least one anti-cancer agent. Certain embodiments contemplate the evaluation after delivering said candidate agents. In some embodiments, the evaluation comprises excising at least one portion of the tissue after introducing said one or more fluid agents. In certain other embodiments, the evaluation comprises imaging the tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 illustrates a method of administering a fluid agent to a tissue.

FIG. 2 is a schematic diagram of a needle array assembly for injecting fluid agents into a biological tissue according to various embodiments.

FIG. 3 is a diagrammatic view of a delivery assembly according to an embodiment.

FIG. 4 shows a diagram of a needle array, according to an embodiment.

FIG. 5 shows elements of a delivery assembly according to another embodiment.

FIG. 6 shows diagrammatically a portion of a tumor illustrating principles of the invention.

FIG. 7 is a diagram of a data processing system according to an embodiment.

FIG. 8 illustrates a slice of lymphoma tumor that was administered doxorubicin via a porous tube of the invention.

FIG. 9 illustrates microscopy of spatially-restricted cell-kill at multiple tumor depths.

FIG. 10 illustrates fluorescent microscopy of spatially-restricted injections of four different amounts of a fluorescent dye into a tumor.

FIG. 11 illustrates ex vivo response to hedgehog pathway antagonism in a human medulloblastoma sample.

FIG. 12 illustrates tumor kill following spatially-restricted injection.

FIG. 13 illustrates Survival of mice injected with vehicle or Shh antagonist.

FIG. 14 illustrates fluorescent imaging of a mouse injected with doxorubicin and control.

FIG. 15 illustrates spatially-restricted lentivirus expression in a tumor.

FIG. 16 illustrates KIF11 shRNA injection in a tumor.

FIG. 17 illustrates cell death in response to shRNA injection in a tumor.

### DETAILED DESCRIPTION

### General Overview

The present invention provides devices and methods for the spatially constrained delivery of one or more fluid agents to a tissue. The disclosure features the use of hollow and/or porous tubes for delivery of fluid agents by diffusion through pores, allowing for control of delivery by varying such parameters as pore diameter. The control of delivery parameters such as localization and rate of delivery provides many advantages in various applications.

In some preferred embodiments, the hollow and/or porous tubes are inserted along parallel axes within a tissue through the use of an array of precisely positioned delivery needles, coupled to an actuator module such as a plunger, allowing for controlled insertion of multiple porous tubes each containing a distinct fluid agent along parallel axes within a tissue. In some cases, passive delivery by diffusion through pores in the porous tube occurs following insertion.

In some preferred cases, the tubes are hollow cylindrical tubes containing an inner reservoir termed the lumen enclosed by tube walls. The walls of a hollow tube may comprise a porous polymer, allowing for release or absorption of a fluid agent by diffusion through the pores. In some cases, the tube is not hollow and comprises a porous polymer capable of absorbing, storing, and releasing a fluid agent. In some cases, the tubes comprise a hydrogel, a term used to describe a network of hydrophilic polymer chains. These polymers are sometimes found as a colloidal gel in which water is the dispersion medium, and can comprise more than 50%, 60%, 70%, 80%, 90%, 95%, 97%, or 99% water. The pore size of a colloidal gel can be controlled, and in some cases, the average pore size of the hydrogel is less than about 20, 30, 40, 50, 100, 200, 500, 1000, 2000, 5000, or 10000 nanometers. In some preferred cases, a rate of diffusion of fluid from a porous tube to a surrounding tissue may be controlled by pore size.

In some preferred embodiments, one or more hollow and/or porous tubes are inserted in a target tissue of an organism through the use of a delivery device, such as a needle. In some cases, more than 1, 2, 3, 4, 5, 6, 10, 20, 50, 100, or 1,000 needles are arranged in an array, such that the needles deliver a plurality of hollow and/or porous tubes to a plurality of parallel regions within the tissue. Each needle may contain one or more porous tubes, or may contain a plurality of porous tubes forming a cylindrical bundle. Upon insertion, two or more porous tubes or two or more bundles of porous tubes may occupy parallel columns in the tissue, with these columns determined by the size, shape, and configuration of the needles. In some preferred cases, solid delivery from these tubes is constrained to a column within the tissue by pore size and diffusion rate.

In some preferred embodiments, the one or more needles are not permeable to the one or more fluid agent. In a further embodiment, the needles are not porous needles or have no pores along the length of the needle.

In some embodiments, a hollow and/or porous tube comprises an indicator particle, a term that refers to a particle capable of transmitting information, including, but not limited to, information about position of delivery or a local response. Non-limiting examples of indicator particles known in the art are a metallic particles, fluorescent dyes, quantum dots, quantum barcodes, radiographic contrast agents, and magnetic resonance imaging contrast agents.

In some embodiments, an indicator particle comprises a quantum dot or a quantum barcode. Methods for manufacturing mondisperse quantum dots and quantum barcodes are known by persons having skill in the art. For example, quantum dots can be synthesized colloidally from precursor compounds dissolved in solutions based on a three component system composed of: precursors, organic surfactants, and solvents. Further discussion of colloidal synthesis of quantum dots can be found in “Colloidal synthesis of nanocrystals and nanocrystal superlattices,” IBM J. Res. & Dev. vol. 45, No. 1, January 2001, pp. 47-56 by C. B. Murray, which is incorporated herein by specific reference in its entirety. A quantum dot typically consists of a semiconductor nanocrystal (e.g., CdSe) surrounded by a passivation shell (e.g., ZnS). Upon absorption of a photon, an electron-hole pair is generated, the recombination of which in −10-20 ns leads to the emission of a less-energetic photon. This energy, and therefore the wavelength, is dependent on the size of the quantum dot particle (smaller particles emit at a lower wavelength), which can be varied almost at will by controlled synthesis conditions.

A quantum barcode has properties similar to a quantum dot except that it absorbs a broad spectrum of light and emits a specific pattern of wavelengths that acts as a particular signature or “barcode.” Typically, a quantum barcode is several different types of quantum dots, each having a particular emission spectrum, that are arranged in a multi layered shell or side-by-side fashion. Quantum barcodes are advantageous at least insofar as their emission pattern produces a particular signature that can be easily detected and tracked.

Metallic particles of a number of types can be incorporated into the porous tubes of the disclosure either by providing metallic nanoparticles particles or preparing them in situ and coating them with one or more of the a polymer materials as discussed herein. Suitable examples of metallic particles that are useful as indicator particles include, but are not limited to, ferric iron oxide (Fe₂O₃) and/or other ferric iron compounds, gadolinium metal or gadolinium-containing compounds, barium sulfate (BaSO₄), or nanogold particles, and combinations thereof.

In some embodiments, a nanoparticle includes a detectable label that is a radiographic contrast agent. Radiographic contrast agents can, for example, allow for x-ray imaging of tissues. In the presently disclosed embodiments, a radiographic contrast agent is included to permit medical personnel to distinguish between cancerous and healthy gastric tissue, as well as indicate treatment response. Suitable examples of radiographic contrast agents that can be incorporated into nanoparticles include, but are not limited to, barium sulfate (BaSO₄) nanoparticles, nanogold particles, iodine-based x-ray contrast agents, and other materials that include heavy nuclei that efficiently absorb x-rays.

In some embodiments, hollow and/or porous tubes comprise a detectable label that is a nuclear magnetic resonance imaging (MRI) contrast agent. While MRI is typically quite useful for imaging tissues, the use of contrast agents is common when imaging the GI tract because it can be difficult to distinguish between the GI tract and the other abdominal organs. In the presently disclosed embodiments, MRI contrast agents are included to permit medical personnel to distinguish between cancerous and healthy gastric tissue. Suitable examples of MRI contrast agents include, but are not limited to, ferric iron oxide (Fe₂O₃) and/or other ferric iron compounds, gadolinium metal or gadolinium-containing compounds, materials containing protons in —CH₂— groups, and compounds containing MRI active nuclei that are not naturally abundant in the body, such as helium-3, carbon-13, fluorine-19, oxygen-17, sodium-23, phosphorus-31, and xenon-129.

Ferric iron and gadolinium compounds are paramagnetic agents that shorten the proton spin relaxation times in surrounding water molecules. Materials containing protons in —CH₂— groups relax at a faster rate than in water resulting in detectable change in the MRI signal. In some embodiments, a porous tube comprises a polymeric material rich in protons in —CH₂— groups, allowing the porous tubes to act as an MRI contrast agent.

In some embodiments, molecules of a receptor-specific ligand are coupled to the nanotracer. In one embodiment, each nanotracer includes functional groups for attachment of receptor-specific ligands thereto. That is, a hollow and/or porous tube may comprise polymeric material that may contain functional groups that provide sites for the attachment of receptor-specific ligands desirable for binding to ligand receptors on the cancerous tissue. Suitable examples of functional groups include, but are not limited to, one or more members selected from the group of a hydroxyl, a carboxyl, a carbonyl, an amine, an amide, a nitrile, a nitrogen with a free lone pair of electrons, an amino acid, a thiol, imidazole, phosphonic acid, phosphinic acid, a sulfonic acid, a sulfonyl halide, or an acyl halide.

Spatial restriction allows for parallel screening of multiple candidate therapeutic agents in a tissue, and controls for tissue heterogeneity by introducing each of the candidate therapeutic agents across an axis in the tissue. In certain embodiments, the selected region of tissue is a portion of a solid tissue in a subject, and in certain further embodiments the subject is one of a preclinical model and a human subject. In certain other embodiments, the method comprises excising at least the portion of the tissue after the introducing. In some embodiments, at least one portion of the tissue has previously been excised. Certain further embodiments comprise at least one of imaging the tissue prior to the excising, imaging the tissue concurrently with the excising, and imaging the tissue after to the excising. In certain other embodiments, the excising comprises excising at least one portion of the tissue at a time that is a selected period of time after introducing one or more fluid agents. The selected period of time may be a range between about 1 minute and 96 hours. In certain embodiments, the selected period of time is a period exceeding one week. In some embodiments, the selected period of time is between one week and six months. Following excision, spatially constrained delivery of multiple candidate agents may allow for ex vivo analysis of the relative efficacies of the agents.

Some embodiments as disclosed herein relate to a method for constrained delivery of a fluid-phase agent to a solid tissue. Such selective delivery obviates the need for excessive systemic concentrations of therapeutic or candidate agents in order to achieve effective concentrations in the desired solid tissue, thereby avoiding detrimental toxicities to uninvolved tissues and also avoiding undesirable side-effects. In other words, the fluid agents can be delivered at or below systematically detectable concentration to achieve an effect in the solid tissue. In some embodiments, the one or more fluid agents have been pre-delivered to the tissue.

In some embodiments, the present method is directed to testing and delivering cancer therapies, where multiple candidate therapeutic agents are delivered along parallel axes of a tumor by insertion of multiple porous tubes into the tumor. Such methods permit efficient pre-clinical and clinical studies of candidate oncology medicines, and facilitate identification of therapeutics having a high likelihood of benefitting individual subjects. The disclosure provides for methods useful in evaluating treatment for cancer and permits early exclusion from a screening program or a therapeutic regimen of candidate drugs to which disease cells can be resistant.

Furthermore, the present disclosure provides for the screening of candidate agents in vivo, allowing advantages over in vitro methods that do not accurately replicate the microenvironment of a solid tissue within a living organism.

### Target Tissues

In some embodiments, the present disclosure exemplifies a system for screening candidate therapeutic agents in a solid tissue. Solid tissues are well known to the medical arts and may include any cohesive, spatially discrete non-fluid defined anatomic compartment that is substantially the product of multicellular, intercellular, tissue and/or organ architecture, such as a three-dimensionally defined compartment that may comprise or derive its structural integrity from associated connective tissue and may be separated from other body areas by a thin membrane (e.g., meningeal membrane, pericardial membrane, pleural membrane, mucosal membrane, basement membrane, omentum, organ-encapsulating membrane, or the like). Non-limiting exemplary solid tissues may include brain, liver, lung, kidney, prostate, ovary, spleen, lymph node (including tonsil), thyroid, pancreas, heart, skeletal muscle, intestine, larynx, esophagus and stomach. Anatomical locations, morphological properties, histological characterization, and invasive and/or non-invasive access to these and other solid tissues are all well known to those familiar with the relevant arts. In some embodiments, the tissue is normal. In some embodiments, the tissue is, or is suspected of being, cancerous, inflamed, infected, atrophied, numb, in seizure, or coagulated. In some embodiments, the tissue is, or is suspected of being, cancerous. In some embodiments, the tissue is cancerous.

In a preferred embodiment, the present method is directed to cancer, and the target tissue comprises a tumor, which may be benign or malignant, and comprises at least one cancer cell selected from the group consisting of a leukemia cell, a pancreatic cancer cell, a prostate cancer cell, a breast cancer cell, a colon cancer cell, a lung cancer cell, a brain cancer cell, a glioma cancer cell, a melanoma cell, a renal cancer cell, and an ovarian cancer cell. In certain embodiments the tumor comprises a cancer selected from adenoma, adenocarcinoma, squamous cell carcinoma, basal cell carcinoma, small cell carcinoma, large cell undifferentiated carcinoma, chondrosarcoma and fibrosarcoma. Art-accepted clinical diagnostic criteria have been established for these and other cancer types, such as those promulgated by the U.S. National Cancer Institute (Bethesda, Md., USA) or as described in DeVita, Hellman, and Rosenberg's Cancer: Principles and Practice of Oncology (2008, Lippincott, Williams and Wilkins, Philadelphia/Ovid, New York); Pizzo and Poplack, Principles and Practice of 25 Pediatric Oncology (Fourth edition, 2001, Lippincott, Williams and Wilkins, Philadelphia/Ovid, New York); and Vogelstein and Kinzler, The Genetic Basis of Human Cancer (Second edition, 2002, McGraw Hill Professional, New York). Other non-limiting examples of typing and characterization of particular cancers are described, e.g., in Ignatiadis et al. (2008 PathobioL 75:104); Curr. Drug Discov. Technol. 5:9); and Auman et al. (2008 Drug Metab. Rev. 40:303). In certain embodiments the selected region of tissue is a portion of a tumor in a subject, and in certain further embodiments the subject is one of a preclinical model and a human patient.

Certain preferred embodiments contemplate a subject or biological source that is a human subject such as a patient that has been diagnosed as having or being at risk for developing or acquiring cancer according to art-accepted clinical diagnostic criteria, such as those of the U.S. National Cancer Institute (Bethesda, Md., USA) or as described in DeVita, Hellman, and Rosenberg's Cancer: Principles and Practice of Oncology (2008, Lippincott, Williams and Wilkins, Philadelphia/Ovid, New York); Pizzo and Poplack, Principles and Practice of Pediatric Oncology (Fourth edition, 2001, Lippincott, Williams and Wilkins, Philadelphia/Ovid, New York); and Vogelstein and Kinzler, The Genetic Basis of Human Cancer (Second edition, 2002, McGraw Hill Professional, New York); certain embodiments contemplate a human subject that is known to be free of a risk for having, developing or acquiring cancer by such criteria.

Certain other embodiments contemplate a non-human subject or biological source, for example a non-human primate such as a macaque, chimpanzee, gorilla, vervet, orangutan, baboon or other non-human primate, including such non-human subjects that may be known to the art as preclinical models, including preclinical models for solid tumors and/or other cancers. Certain other embodiments contemplate a non-human subject that is a mammal, for example, a mouse, rat, rabbit, pig, sheep, horse, bovine, goat, gerbil, hamster, guinea pig or other mammal; many such mammals may be subjects that are known to the art as preclinical models for certain diseases or disorders, including solid tumors and/or other cancers (e.g., Talmadge et al., 2007 Am. J. Pathol. 170:793; Kerbel, 2003 Canc. Biol. Therap. 2(4 Suppl 1):5134; Man et al., 2007 Canc. Met. Rev. 26:737; Cespedes et al., 2006 Clin. TransL Oncol. 8:318). The range of embodiments is not intended to be so limited, however, such that there are also contemplated other embodiments in which the subject or biological source may be a non-mammalian vertebrate, for example, another higher vertebrate, or an avian, amphibian or reptilian species, or another subject or biological source. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells). In certain embodiments of the present invention, the tissue of a transgenic animal may be targeted. In some embodiments, the solid tissue is a xenograft produced by introducing one or more cells of one organism (e.g. cultured human cancer cells) into a nonhuman model organism.

In some embodiments, the subject is a preclinical animal model. In some preferred embodiments, the subject is one of a mouse model or rat model. A preclinical model may be an animal model that is the recipient of a xenograft or xenotransplantation, terms that are used interchangeably to refer to the transplantation of living cells, tissues or organs from one species to another. In some preferred cases, the preclinical model is the recipient of one or more cancer cells that develops into a tumor. The recipient preclinical model may be an immunocompromised animal, such as a SCID mouse or nude mouse. An athymic nude mouse is a laboratory mouse from a strain with a genetic mutation that causes a deteriorated or absent thymus, resulting in an inhibited immune system due to a greatly reduced number of T cells. An immunocompromised state in a preclinical model may be the result of genetic abnormalities, or it may be the result of drug treatments to suppress immune system function. Immunosuppressive drugs or immunosuppressive agents are drugs that inhibit or prevent activity of the immune system. Non-limiting examples of immunosuppressive drugs include glucocorticoids; cytostatics; alkylating agents; antimetabolites including folic acid analogues, such as methotrexate, and purine analogues such as azathioprine and mercaptopurine; azathioprine and mercaptopurine; cytotoxic antibiotics, including dactinomycin, anthracyclines, mitomycin C, bleomycin, and mithramycin; polyclonal and monoclonal antibodies targeting elements of the immune system; and drugs acting on immunophilins, including cyclosporin, tacrolimus, voclosporin and other calcineurin inhibitors, and sirolimus; interferons, opioids, TNF-binding proteins, mycophenolate, and fingolimod.

In some embodiments, the solid tissue is soft tissue. Non-limiting examples of soft tissue include muscle, adipose, skin, tendons, ligaments, blood, and nervous tissue. Biological samples can be provided by obtaining a blood sample, biopsy specimen, tissue explant, organ culture, biological fluid or any other tissue or cell preparation from a subject or a biological source.

The subject or biological source can be a human or non-human animal, a transgenic or cloned or tissue-engineered (including through the use of stem cells) organism, a primary cell culture or culture adapted cell line including but not limited to genetically engineered cell lines that can contain chromosomally integrated or episomal recombinant nucleic acid sequences, immortalized or immortalizable cell lines, somatic cell hybrid cell lines, differentiated or differentiatable cell lines, transformed cell lines and the like. In some embodiments of the invention, the subject or biological source can be suspected of having or being at risk for having a malignant condition, and in some embodiments of the invention the subject or biological source can be known to be free of a risk or presence of such disease.

### Fluid Agents

In certain embodiments, the fluid agent comprises an agent that is selected from (a) a gene therapy agent; (b) a chemotherapy agent; (c) a small molecule; (d) an antibody; (e) a protein; (f) one of a small interfering RNA and an encoding polynucleotide therefor; (g) one of an antisense RNA and an encoding polynucleotide therefor; (h) one of a ribozyme and an encoding polynucleotide therefor; (i) a detectable label; (j) one of a therapeutic protein, polypeptide, and a peptidomimetic; (k) a microRNA (miRNA); and (k) a drug. In certain further embodiments, the detectable label may be selected from a radiolabel, a radio-opaque label, a fluorescent label, a colorimetric label, a dye, an enzymatic label, a GCMS tag, avidin, and biotin. In some embodiments, the drug refers to any FDA approved drug, any drug currently in clinical trials, and any drug failed in clinical trials. In certain further embodiments, the drug is an anti-cancer agent.

In some embodiments, the fluid agent is selected from (i) a gene therapy agent that comprises at least one operably linked promoter; (ii) a small interfering RNA-encoding polynucleotide that comprises at least one operably linked promoter; (iii) an antisense RNA encoding polynucleotide that comprises at least one operably linked promoter; and (iv) a ribozyme-encoding polynucleotide that comprises at least one operably linked promoter. In certain further embodiments, the operably linked promoter is selected from a constitutive promoter and a regulatable promoter. In certain still further embodiments, the regulatable promoter is selected from an inducible promoter, a tightly regulated promoter and a tissue-specific promoter.

Candidate agent or candidate compound may be used interchangeably with fluid agent. Candidate agent or candidate compound refers to any fluid or molecule in an aqueous solution, mixture, or colloid that may be delivered to a target tissue. When used to refer to agents delivered from needles, the term fluid is to be read broadly to read on any substance capable of flowing through such a needle, including liquids, gases, colloids, suspended solids, etc.

Selection of candidate oncology agents is understood and determinable by one skilled in the relevant arts (see, e.g., Berkow et al., eds., The Merck Manual, 16^{th} edition, Merck and Co., Rahway; N.J., 1992; Goodman et al., eds., Goodman and Gilman's The Pharmacological Basis of Therapeutics, 10^{th} edition, Pergamon Press, Inc., Elmsford, N.Y., (2001); De Vita, Hellman, and Rosenberg's Cancer: Principles and Practice of Oncology (2008, Lippincott, Williams and Wilkins, Philadelphia/Ovid, New York); Pizzo and Poplack, Principles and Practice of Pediatric Oncology (Fourth edition, 2001, Lippincott, Williams and Wilkins, Philadelphia/Ovid, New York); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)). Candidate agents can be selected from resources that disclose listings of investigational therapeutics, for instance, the National Institutes of Health (Bethesda, Md.) which maintains a database of ongoing and planned clinical trials at its “ClinicalTrials.gov” website.

Candidate agents for use in screening methods and in methods of rating candidate agents for development into therapeutic agents such as a therapeutic agent for treating a solid tumor can be provided as “libraries” or collections of compounds, compositions or molecules. Such molecules typically include compounds known in the art as “small molecules” and having molecular weights less than 10⁵ Daltons, less than 10⁴ Daltons, or less than 10³ Daltons.

For example, a plurality of members of a library of test compounds can be introduced as candidate agents to a region of a solid tumor of known tumor type in each one or a plurality of subjects having a tumor of the known tumor type, by distributing each of the candidate agents to a plurality of positions along an axis within the region in each subject, and after a selected period of time (e.g., a range of time, a minimum time period or a specific time period) the region of solid tumor in which the candidate agents have been introduced can be imaged or removed from each subject, and each region compared by detecting an effect (if any) of each agent on the respective position within the region, for instance, by determining whether an altered physiologic state is present as provided herein, relative to positions in the region that are treated with control agents as provided herein, which would either produce no effect (negative control) or a readily detectable effect (positive control).

Candidate agents further can be provided as members of a combinatorial library, which can include synthetic agents prepared according to a plurality of predetermined chemical reactions performed in a plurality of reaction vessels. For example, various starting compounds can be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that can include small molecules as provided herein (see e.g., PCT/US94/08542, EP 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries can be prepared according to established procedures, and tested for their influence on an indicator of altered mitochondrial function, according to the present disclosure.

Other candidate agents can be proteins (including therapeutic proteins), peptides, peptidomimetics, polypeptides, and gene therapy agents (e.g., plasmids, viral vectors, artificial chromosomes and the like containing therapeutic genes or polynucleotides encoding therapeutic products, including coding sequences for small interfering RNA (siRNA), ribozymes and antisense RNA) which in certain further embodiments can comprise an operably linked promoter such as a constitutive promoter or a regulatable promoter, such as an inducible promoter (e.g., IPTG-inducible), a tightly regulated promoter (e.g., a promoter that permits little or no detectable transcription in the absence of its cognate inducer or derepressor) or a tissue-specific promoter. Methodologies for preparing, testing and using these and related agents are known in the art. See, e.g., Ausubel (Ed.), Current Protocols in Molecular Biology (2007 John Wiley & Sons, NY); Rosenzweig and Nabel (Eds), Current Protocols in Human Genetics (esp. Ch. 13 therein, “Delivery Systems for Gene Therapy”, 2008 John Wiley & Sons, NY); Abell, Advances in Amino Acid Mimetics and Peptidomimetics, 1997 Elsevier, N.Y.

Other candidate agents can be antibodies, including naturally occurring, immunologically elicited, chimeric, humanized, recombinant, and other engineered antigen-specific immunoglobulins and artificially generated antigen-binding fragments and derivatives thereof, such as single-chain antibodies, minibodies, Fab fragments, bi-specific antibodies and the like. See, e.g., Coligan et al. (Eds.), Current Protocols in Immunology (2007 John Wiley & Sons, NY); Harlow and Lane, Antibodies: A Laboratory Manual (1988 Cold Spring Harbor Press, Cold Spring Harbor, N.Y.); Harlow and Lane, Using Antibodies (1999 Cold Spring Harbor Press, Cold Spring Harbor, N.Y.).

Pharmaceutically acceptable carriers for therapeutic use are well known in the pharmaceutical art, and are described, for example, in Remingtons Pharmaceutical Sciences. Mack Publishing Co. (A. R. Gennaro edit. 1985). For example, sterile saline and phosphate-buffered saline at physiological pH can be used. Preservatives, stabilizers, dyes and other ancillary agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id. “Pharmaceutically acceptable salt” refers to salts of drug compounds derived from the combination of such compounds and an organic or inorganic acid (acid addition salts) or an organic or inorganic base (base addition salts). The agents, including drugs, contemplated for use herein can be used in either the free base or salt forms, with both forms being considered as being within the scope of the certain present invention embodiments.

The pharmaceutical compositions that contain one or more agents can be in any form which allows for the composition to be administered to a subject. According to some embodiments the composition will be in liquid form and the route of administration will comprise administration to a solid tissue as described herein. The term parenteral as used herein includes transcutaneous or subcutaneous injections, and intramuscular, intramedullar and intrastemal techniques.

The pharmaceutical composition is formulated so as to allow the active ingredients contained therein to be bioavailable upon administration of the composition to a subject such as a human subject. Compositions that will be administered to a subject can take the form of one or more doses or dosage units, where for example, a pre-measured fluid volume can comprise a single dosage unit, and a container of one or more compositions (e.g., drugs) in liquid form can hold a plurality of dosage units. A dose of a drug includes all or a portion of a therapeutically effective amount of a particular drug that is to be administered in a manner and over a time sufficient to attain or maintain a desired concentration range of the drug, for instance, a desired concentration range of the drug in the immediate vicinity of a delivery needle in a solid tissue, and where the absolute amount of the drug that comprises a dose will vary according to the drug, the subject, the solid tissue and other criteria with which the skilled practitioner will be familiar in view of the state of the medical and pharmaceutical and related arts. In certain embodiments at least two doses of the drug can be administered, and in certain other embodiments 3, 4, 5, 6, 7, 8, 9, 10 or more doses can be administered.

A liquid pharmaceutical composition as used herein, whether in the form of a solution, suspension or other like form, can include one or more of the following adjuvants: sterile diluents such as water for injection, saline solution, physiological saline, Ringer's solution, saline solution (e.g., normal saline, or isotonic, hypotonic or hypertonic sodium chloride), fixed oils such as synthetic mono or digylcerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. In some embodiments, physiological saline is the adjuvant. An injectable pharmaceutical composition can be sterile. It can also be desirable to include other components in the preparation, such as delivery vehicles including but not limited to aluminum salts, water-in-oil emulsions, biodegradable oil vehicles, oil-in-water emulsions, biodegradable microcapsules, hydrogels, and liposomes.

While any suitable carrier known to those of ordinary skill in the art can be employed in the pharmaceutical compositions of this invention, the type of carrier will vary depending on the mode of administration and whether a conventional sustained drug release is also desired. For parenteral administration, such as supplemental injection of drug, the carrier can comprise water, saline, alcohol, a fat, a wax or a buffer. Biodegradable microspheres (e.g., polylactic galactide) can also be employed as carders for the pharmaceutical compositions of this invention. Suitable biodegradable microspheres are disclosed, for example, in U.S. Pat. Nos. 4,897,268 and 5,075,109. In some embodiments, the microsphere be larger than approximately 25 microns, while other embodiments are not so limited and contemplate other dimensions.

Pharmaceutical compositions can also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. In some embodiments, an agent (e.g., a therapeutic drug or a candidate drug) is formulated as a lyophilizate using appropriate excipient solutions (e.g., sucrose) as diluents.

Certain embodiments contemplate direct delivery of multiple drugs, candidate drugs, imaging agents, positional markers, indicators of efficacy and appropriate control compositions to a plurality of spatially defined locations along parallel axes in a solid tissue, such as a solid tumor, followed, after a desired time interval, by excision of the treated tissue and evaluation or analysis of the tissue for effects of the treatments. Indicators of efficacy can be, for example, detectable indicator compounds, nanoparticles, nanostructures or other compositions that comprise a reporter molecule which provides a detectable signal indicating the physiological status of a cell, such as a vital dye (e.g., Trypan blue), a colorimetric pH indicator, a fluorescent compound that can exhibit distinct fluorescence as a function of any of a number of cellular physiological parameters (e.g., pH, intracellular Ca²⁺ or other physiologically relevant ion concentration, mitochondrial membrane potential, plasma membrane potential, etc., see Haugland, The Handbook: A Guide to Fluorescent Probes and Labeling Technologies (10th Ed.) 2005, Invitrogen Corp., Carlsbad, Calif.), an enzyme substrate, a specific oligonucleotide probe, a reporter gene, or the like. Control compositions can be, for example, negative controls that have been previously demonstrated to cause no statistically significant alteration of physiological state, such as sham injection, saline, DMSO or other vehicle or buffer control, inactive enantiomers, scrambled peptides or nucleotides, etc.; and positive controls that have been previously demonstrated to cause a statistically significant alteration of physiological state, such as an FDA-approved therapeutic compound.

In some embodiments, the fluid agent further comprises a dye. The dye can be imaged after administration of the pharmaceutical composition to a solid tissue to observe the distribution and activity of a therapeutic agent present in the same pharmaceutical composition. In some embodiments, the dye is a fluorescent dye. In some embodiments, the dye is a radioactive dye.

In some embodiments, the fluid agent comprises a positional marker. Positional markers are known and include, as non-limiting examples, fluorescent quantum dots, India ink, metal or plastic beads, dyes, stains, tumor paint (Veiseh et al., 2007 Canc. Res. 67:6882) or other positional markers, and can be introduced at desired positions. Markers can include any subsequently locatable source of a detectable signal, which can be a visible, optical, colorimetric, dye, enzymatic, GCMS tag, avidin, biotin, radiological (including radioactive radiolabel and radio-opaque), fluorescent or other detectable signal.

A detectable marker thus comprises a unique and readily identifiable gas chromatography/mass spectrometry (GCMS) tag molecule. Numerous such GCMS tag molecules are known to the art and can be selected for use alone or in combination as detectable identifier moieties. By way of illustration and not limitation, various different combinations of one, two or more such GCMS tags can be added to individual reservoirs of the device described herein in a manner that permits the contents of each reservoir to be identified on the basis of a unique GCMS “signature”, thereby permitting any sample that is subsequently recovered from an injection region to be traced back to its needle of origin for identification purposes. Examples of GCMS tags include α,α,α-trifluorotoluene, α-methylstyrene, o-anisidine, any of a number of distinct cocaine analogues or other GCMS tag compounds having readily identifiable GCMS signatures under defined conditions, for instance, as are available from SPEX CertiPrep Inc. (Metuchen, N.J.) or from SigmaAldrich (St. Louis, Mo.), including Supelco® products described in the Supelco® 2005 gas chromatography catalog and available from SigmaAldrich.

### Porous Tubes

The term hollow and/or porous tube and porous tube can be used interchangeably. The present method provides for the administration of a fluid agent to a tissue through the use of one or more hollow and/or porous tubes. The fluid agent contacts the tissue by diffusion through pores of the tubes. Porous tubes used in the devices and methods of the present application may be hollow, or may uniformly comprise porous material. The porous tubes are suitable for containing, storing, administering, delivering, and transporting contents. The contents can be a pharmaceutical composition comprising one or more candidate agents. The candidate agents within a single hollow and/or porous tube can be the same or can be a mixture of different types of candidate agents. Within a plurality of hollow and/or porous tubes, each tube can contain the same candidate agents as another tube, or different candidate agents as another tube. In some embodiments, every hollow and/or porous tube contains candidate agents that are unique from the candidate agents contained in every other tube of the plurality of tubes.

The hollow and/or porous tubes can be connected to a frame that holds the tubes and facilitates drug delivery. The hollow and/or porous tubes can be detachable from the frame. The number and spatial orientation of hollow and/or porous tubes connected to the frame can be varied based on the drug-delivery needs of a subject.

A hollow and/or porous tube is made of a tube material. The tube material is suitable for containing, storing, administering, delivering, and transporting a fluid agent. The fluid agent can be a pharmaceutical composition comprising one or more therapeutic agents. In some embodiments, the tube material is essentially inert to acid. In some embodiments, the tube material is essentially inert to base. In some embodiments, the tube material is essentially inert to acid and base. In some embodiments, the tube material is insoluble in water. In some embodiments, the tube material is insoluble in organic solvents. In some embodiments, the tube material is essentially insoluble in organic solvents. In some embodiments, the tube material is insoluble in non-halogenated organic solvents. In some embodiments, the tube material is essentially insoluble in non-halogenated organic solvents.

The tube material is biocompatible. The tube material is essentially physiologically-inactive, and does not trigger physiological events. The tube material does not cause inflammation, immune response, infection, or any other sort of rejection within a solid tissue. In some embodiments, the tube material is biodegradable. Biodegradable materials include synthetic biodegradable polymers and naturally occurring biodegradable polymers. Examples of synthetic biodegradable polymers include but not limited to polyalkene esters, polylactic acid and its copolymers, polyamide esters, polyvinyl esters, polyvinyl alcohols, and polyanhydrides. Examples of naturally occurring biodegradable polymers include but not limited to polysaccharide, for example, starch and cellulose; proteins, for example, gelatin, casein, silk, and wool; polyesters, for example, polyhydroxy alkanoates; and others, for example, lignin, and shella. In some embodiments, the tube material decomposes over time within a solid tissue. The tube material is thermostable, and the tubes can be sterilized in an autoclave prior to use on a subject.

The tube material is suitable for being shaped into a tube, but also suitable for retaining the tube shape upon deposition into solid tissue. The tube material is suitable for being broken, cut, sliced, disjoined, or separated in a clean way, and can be broken, cut, sliced, disjoined, or separated after deposition into a solid tissue. In some embodiments, the tube material is scissile.

In some embodiments, the tube material is polymeric. In some embodiments, the tube material is co-polymeric. In some embodiments, the tube material is a cross-linked polymer or co-polymer. Non-limiting examples of tube materials include polysulfone, polyamine, polyamide, polycarbonate, polycarbamate, polyurethane, polyester, polyether, polyolefin, polyaromatic materials. In some embodiments, the tube material is polysulfone.

The preparation of hollow tubes from polymers can be achieved by various routes. These are referred to as wet, dry or melt-forming processes. Melt-forming involves heating a polymer above its melting point and extruding it through an orifice (usually referred to as a die) which is designed to form a hollow tube. Once extruded, the melt is cooled via a quench which allows the polymer to solidify into a fine tube. In the dry-forming process, a solution of the polymer is extruded through a desired orifice and is fed into a heated column which allows for evaporation of the solvent and subsequent formation of a tube. In a wet-membrane forming process, a solution of the polymer is extruded though an orifice and quenched in a non-solvent for the polymer resulting in coagulation of the polymer to a tube. Of the above mentioned forming processes, wet-membrane forming allows one to easily produce hollow porous tubes. The particular forming process used will be dependent upon the polymer used and type of hollow tube desired.

In some embodiments, the tube material comprises a plurality of pores. The contents of the tube can diffuse from the tube into solid tissue via the pores. The rate of diffusion form the porous tube into the solid tissue can be influenced by the pore size, for example, larger pores result in a higher diffusion rate. In some embodiments, the tube material is permeable. In some embodiments, a porous tube is permeable.

The effective agent diffuses in the direction of lower chemical potential, i.e., toward the exterior surface of the device. At the exterior surface of the device, equilibrium is again established. A steady state flux of the effective agent will be established in accordance with Fick's Law of Diffusion. The rate of passage of the drug through the material by diffusion is generally dependent on the solubility of the drug therein, as well as on the thickness of a porous wall. Selection of porous tube materials may depend on the particular fluid agent to be delivered.

In producing a porous material, the size of the pores is affected by the solvent strength of a polymer. A rapid decrease in solvent strength often tends to entrap a dispersion of small droplets within the continuous polymer phase. A slow decrease in solvent strength allows for nucleation sites within the polymer matrix allowing for formation of larger pores. In such cases, the reduction in solvent strength must be rapid enough to allow for the structure of the membrane to set.

Another way to change porosity and volume of the porous network in producing a porous polymer is to change the concentration of the polymer solution. Lower concentrations have a tendency to promote larger pores and greater pore volume. However, there is a limit to how high (usually no more than 45% w/w) the polymer concentration can be in a solvent. Otherwise, the polymer will become the dispersed phase in a continuous solvent phase, thereby eliminating the porous network. Another method to achieve porous tubular membranes is to cause a rapid phase inversion of the polymer solution by cooling.

In some embodiments, the average pore size is less than about 1, 5, 10, 20, 30, 40, 50, 100, 200, 500, 1000, 2000, 5000, or 10000 nanometers. All the pores of a single tube can be about the same pore size. In some embodiments, each pore of a single tube has a pore size that is independent of the pore size of all the other pores of the tube. Within a plurality of porous tubes, all pores can have about the same pore size, or each pore can have a size that is independent of the size of all the other pores of the plurality of porous tubes.

The permeability of a pore is preferably 200 ml/m²/hr./mmHg or more; 300 ml/m²/hr/mmHg or more; 400 ml/m²/hr./mmHg or more; or 500 ml/m²/hr./mmHg or more. In some embodiments, the coefficient of water permeability is preferably 2,000 ml/m²/hr./mmHg or less; 1,800 ml/m²/hr/mmHg or less; 1,500 ml/m²/hr./mmHg or less; 1,300 ml/m²/hr./mmHg or less; or 1,000 ml/m²/hr./mmHg or less.

Within a single porous tube, the pore sizes can vary by as much as 5%, 10%, 15%, 20%, 25%, 30%, 40%, 50%, 60%, 70%, 75%, 80%, 90%, 100%, 125%, 150%, 200%, 300%, 400%, 500%, 750%, or 1,000%. Within a single porous tube, the pore sizes can vary by as much as about 5%, about 10%, about 15%, about 20%, about 25%, about 30%, about 40%, about 50%, about 60%, about 70%, about 75%, about 80%, about 90%, about 100%, about 125%, about 150%, about 200%, about 300%, about 400%, about 500%, about 750%, or about 1,000%.

The pore size can control the rate of diffusion, and the pore size can be modulated to control the rate of diffusion. A porous tube can be generated having a pre-determined average pore size for the purpose of controlling the rate of diffusion. Different pharmaceutical compositions of therapeutic agents can diffuse form the porous tubes at varying rates, controlled in part by the physical and chemical properties of the pharmaceutical compositions, therapeutic agents, and porous tube materials. Porous tubes with varying average pore sizes can be generated and used experimentally to find a pore size that provides a desired diffusion rate for a specific pharmaceutical composition or therapeutic agent.

In some embodiments, the entire tube contains a fluid agent, such as a pharmaceutical composition or therapeutic agents. In some embodiments, a porous tube has a top end and bottom end, and a bottom end contains a fluid agent, such as a pharmaceutical composition or therapeutic agents, while the top end does not contain a fluid agent, such as a pharmaceutical composition or therapeutic agents. The bottom end of a tube can be attached to a device suitable for assisting in the administration of the contents into a solid tissue. The bottom end of a tube can be connected, for example, to a needle, port, catheter, intravenous line, or other apparatus suitable for delivering a pharmaceutical composition into a solid tissue. In some embodiments, the apparatus (e.g., a needle) is suitable for penetrating a solid tissue.

The top end of a tube can be attached to a device suitable for assisting in the administration of the contents into a solid tissue. The top end of a tube can be connected, for example, to a plunger, pump, piston, or other apparatus suitable for providing a pressure sufficient to deliver a pharmaceutical composition into a solid tissue, or any such device described herein.

The tubes can be loaded, packed, or charged with a fluid agent. The tubes can be loaded immediately prior to use, or can be loaded, stored, and shipped. Either end of a porous tube, or both ends, may be sealed following loading with a fluid agent. In some cases, one or both ends of a porous tube may be sealed prior to loading with a fluid agent by, for example, soaking the tube for an extended period of time in the fluid agent. A porous tube can be charged with a fluid agent by passive diffusion (e.g. osmosis).

The narrow diameter and shape of the tubes provides for convenient loading by capillary action. A tube, or a plurality thereof, can be dipped into a fluid pharmaceutical composition, and the pharmaceutical composition can be drawn into the tubes. In a closed environment, the application of positive pressure to the pharmaceutical composition results in loading a greater amount of the pharmaceutical composition into the tubes; thus, the amount of pharmaceutical formulation in a tube can be controlled easily and reliably.

The porosity of the tubes can provide for convenient loading by soaking the tubes in a bath of a fluid pharmaceutical composition. The pharmaceutical composition can diffuse into the tubes, for example, through the pores or via permeability of the tube material. The amount of pharmaceutical composition that diffuses into the tubes can be influenced, for example, by external pressure, pore size, permeability, tube length, bath depth, bath amount, amount of time spent in the bath, and tube material.

A pharmaceutical composition loaded into a hollow and/or porous tube comprises one or more candidate agents. Non-limiting examples of candidate agents compatible with the invention are detailed elsewhere herein and include anti-cancer agents, anti-inflammatory agents, anti-infective agents, regenerative agents, relaxing agents, apoptosis-inhibiting agents, apoptosis-inducing agents, anti-coagulatory agents, dermatological agents, growth-stimulating agents, vasodilating agents, vasorestricting agents, analgesic agents, anti-allergic agents, and any candidate agents described herein. In some embodiments, the candidate agent is an anti-cancer agent.

The hollow and/or porous tubes may comprise the same fluid agent or different fluid agents. In some embodiment, none of the hollow and/or porous tubes comprises the same fluid agent as the agent in any other tubes. In other embodiments, at least two of the porous tubes comprise the same fluid agent. In still other embodiments, at least one porous tube comprises two or more fluid agents.

### Hydrogels

In some embodiments, a porous tube comprises a hydrogel. The hydrogel is effective to slow the rate of diffusion or dispersion of a pharmaceutical formulation through a solid tissue. In some embodiments, a pharmaceutical composition containing the hydrogel disperses through a solid tissue to a lesser degree than does an analogous pharmaceutical composition lacking the hydrogel. In some embodiments, a pharmaceutical composition containing the hydrogel disperses through a solid tissue more slowly than does an analogous pharmaceutical composition lacking the hydrogel. The hydrogel may be one polymer or a mixture of two or more polymers. In some embodiments, the hydrogel comprises a binary mixture of at least one polymer with two different molecular weight ranges. In some embodiment, the hydrogel is PEG. In a further embodiment, the two different molecular ranges comprise 500-2000 and 4,000-8,000. In another further embodiment, the two different molecular ranges comprise 4,000-8,000 and 10,000-45,000. By adjusting the molecular weight and ratio of the binary system, the diffusion rate of the fluid agents can be controlled.

Another property for characterization of hydrogel is tensile strength. The hydrogel may have a tensile strength of at least 20 gf/cm² in the dry state. In a further embodiment, the hydrogel has a tensile strength of 20-120 gf/cm² in the dry state. In some embodiments, the hydrogel may have a tensile strength of at least 5 gf/cm² in the hydrate state. In a further embodiment, the hydrogel has a tensile strength of 5-15 gf/cm² in the hydrate state.

The hydrogel can be present in an amount from about 1% to about 99% of a pharmaceutical composition. In some embodiments, the hydrogel is present in an amount of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a pharmaceutical composition.

Exemplary porous materials suitable for biological use are described in U.S. Pat. No. 4,014,335, which is incorporated herein by reference in its entirety. These materials include cross-linked polyvinyl alcohol, polyolefins or polyvinyl chmorides or cross-linked gelatins; regenerated, insoluble, non-erodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen.

In some embodiments, the hydrogel comprises collagen. Non-limiting examples of sources of collagen include Engelbreth-Holm-Swarm murine sarcoma basement membrane, bovine achilles tendon, bovine nasal septum, bovine tracheal cartilage, calf skin, chicken sternal cartilage, human lung, human placenta, kangaroo tail, mouse sternum, and rat tail tendon. In some cases, collagen may comprise more than 0.1%, 0.2%, 0.5%, 0.7%, 1%, 1.2%, 1.4%, 1.6%, 1.8%, 2%, or 5% of the hydrogel. In some cases, recombinant collagen may be used. Several sources of collagen are described in US Patent Application No. US20070254041. Collagen material that is insoluble in water can be used, and can be derived from natural tissue sources (e.g. xenogenic, allogenic, or autogenic relative to the recipient human or other patient) or recombinantly prepared. Collagens can be subclassified into several different types depending upon their amino acid sequence, carbohydrate content and the presence or absence of disulfide crosslinks. Types I and III collagen are two of the most common subtypes of collagen. Type I collagen is present in skin, tendon and bone, whereas Type III collagen is found primarily in skin. The collagen used in compositions of the invention can be obtained from skin, bone, tendon, or cartilage and purified by methods well known in the art and industry. Alternatively, the collagen can be purchased from commercial sources. Type I bovine collagen is preferred for use in the invention.

The collagen can be atelopeptide collagen and/or telopeptide collagen. Still further, either or both of non-fibrillar and fibrillar collagen can be used. Non-fibrillar collagen is collagen that has been solubilized and has not been reconstituted into its native fibrillar form.

Suitable collagen products are available commercially, including for example from Kensey Nash Corporation (Exton, Pa.), which manufactures a fibrous collagen known as semed F, from bovine hides. Collagen materials derived from bovine hide are also manufactured by Integra Life Science Holding Corporation (Plainsboro, N.J.). Naturally-derived or recombinant human collagen materials are also suitable for use in the invention. Ilustratively, recombinant human collagen products are available from Fibrogen, Inc. (San Francisco, Calif.). The solid particulate collagen incorporated into the inventive compositions can be in the form of intact or reconstituted fibers, or randomly-shaped particles, for example.

Collagen can be dissolved in water to form an aqueous solution at room temperature, but undergoes polymerization to form a gel at 37 degrees. Miyata notes in U.S. Pat. No. 4,164,559 that the chemistry, molecular structure and biochemical properties of collagen have been well established (Annual Review of Biophysics and Bioengineering, Vol. 3, pp. 231-253, 1974). Collagen is a major protein of connective tissue such as cornea, skin, etc., and can be solubilized and purified by the treatment with protelolytic enzymes (other than collagenase) such as pepsin. Solubilized collagen is telopeptides-poor, relatively inexpensive, not antigenic and useful as a biomedical material. Enzyme solubilized native collagen is soluble in acidic pH but polymerizes to form a gel at physiologic pH and at 37 degrees.

In other embodiments, the hydrogel comprises polyethylene glycol (PEG), in various formulations known in the art. Non-limiting examples of polymers that may be present in PEG hydrogels include polylactic acid (PLA), poly(lactic-co-glycolic acid) (PLGA), and polycaprolactone (PCL). Some examples of these copolymers include PLA-PEG-PLA, PLGA-PEG-PLA, and mPEG-b+PCL(1200)-b-PEG(6000)-b−PCL(1200) copolymer. PLGA is a copolymer which is used in a host of Food and Drug Administration (FDA) approved therapeutic devices, owing to its biodegradability and biocompatibility. PLGA is synthesized by means of random ring-opening co-polymerization of two different monomers, the cyclic dimers (1,4-dioxane-2,5-diones) of glycolic acid and lactic acid. Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid). All PLGAs are amorphous rather than crystalline and show a glass transition temperature in the range of 40-60 degrees. There is very minimal systemic toxicity associated with using PLGA for drug delivery or biomaterial applications. PLA is a biodegradable, thermoplastic, aliphatic polyester derived from renewable resources, such as corn starch, tapioca products, or sugarcanes. PCL is a biodegradable polyester with a low melting point of around 60° C. and a glass transition temperature of about −60° C. PCL is prepared by ring opening polymerization of e-caprolactone using a catalyst such as stannous octanoate. PCL is an FDA-approved material that is used in the human body, and undergoes slow degradation upon implantation.

### Delivery Devices and Methods

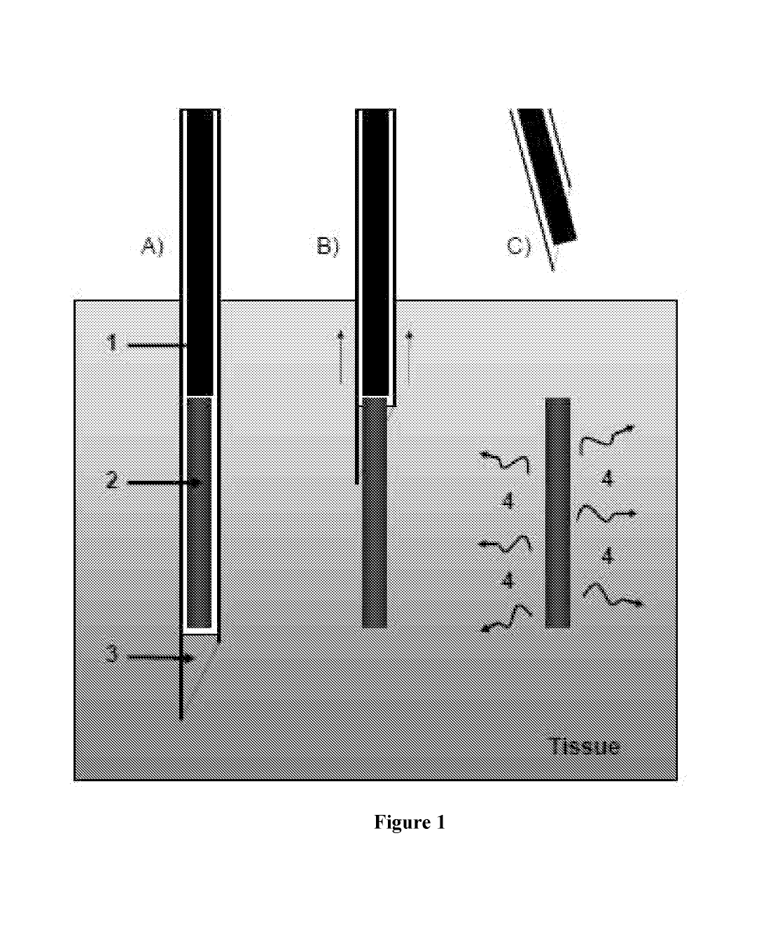

FIG. 1 illustrates delivery of a fluid agent to a tissue through use of a needle and a porous tube. A needle 3 is inserted into a tissue, and a plunger 1 is depressed to inject a porous tube 2 into the tissue. Following insertion into the tissue, a fluid agent 4 is delivered to the tissue by diffusion through pores in the tube.

In some embodiments, one or more porous tubes are inserted into a tissue through use of a carrier device such as a needle. In some preferred embodiments, a plurality of porous tubes is inserted along parallel axes of a tissue through the use of a needle array, such as that described in PCT/US2008/073212, which is hereby incorporated by reference in its entirety.

In some cases, a plurality of needles is attached to a plurality of actuators coupled to a plurality of porous tubes or bundles of porous tubes within a plurality of reservoirs, such that depressing the plunger causes ejection of the porous tubes, or injection of the porous tubes into a tissue. In certain further embodiments the plungers of the plurality of plungers are operatively coupled together at respective second ends so as to be simultaneously depressable. Certain still further embodiments comprise a plunger driver configured to depress all of the plurality of plungers at a selectively variable rate. In other embodiments each of the plurality of actuators comprises one of a plurality of fluid transmission lines having first and second ends, a first end of each of the plurality of fluid transmission lines being coupled to a respective one of the plurality of reservoirs. In other embodiments the device comprises a fluid pressure source, and each of the plurality of actuators comprises a fluid coupling between the fluid pressure source and a respective one of the plurality of reservoirs. In further embodiments the fluid pressure source comprises at least one of a compressor, a vacuum accumulator, a peristaltic pump, a master cylinder, a microfluidic pump, and a valve.

A tube can be partially- or fully-submerged in the tissue. Deposition of tubes into the tissue can be facilitated by inserting the tube into the tissue via a needle. The tube can be further deposited into the tissue by depression of a plunger associated with the needle.

After deposition of the tube into the tissue, the tube can be broken, cut, sliced, disjoined, or separated to remove the top end of the tube from the bottom end of the tube. The bottom end remains deposited in the tissue, whereas the top end is removed from the tissue. In some embodiments, the bottom end of the tube contains a therapeutic agent and the top end of the tube does not contain a therapeutic agent. In some embodiments, both the bottom end and the top end of the tube contain a therapeutic agent.

Once a tube has been deposited into the tissue, the contents of the tube (for example, a pharmaceutical composition or a therapeutic agent) can diffuse from the tube into the tissue. The rate of diffusion can be influenced by the porosity of the tube. The contents can diffuse through the tube material into the tissue over a period of about a minute to about a month; about an minute to about a week; about 12 hours to about 72 hours; or about 24 hours to about 48 hours.

A device for the insertion of one or more porous tubes into a tissue, such as a needle array, may be loaded with the one or more porous tubes prior to attachment to one or more actuators. In some cases, loading of the needle array with porous tubes may occur following attachment to actuators, wherein the actuators are driven to produce negative pressure in the needles, causing them to draw in porous contents (e.g. a fluid agent within a hydrogel), thereby forming a porous tube within the reservoir of a needle.

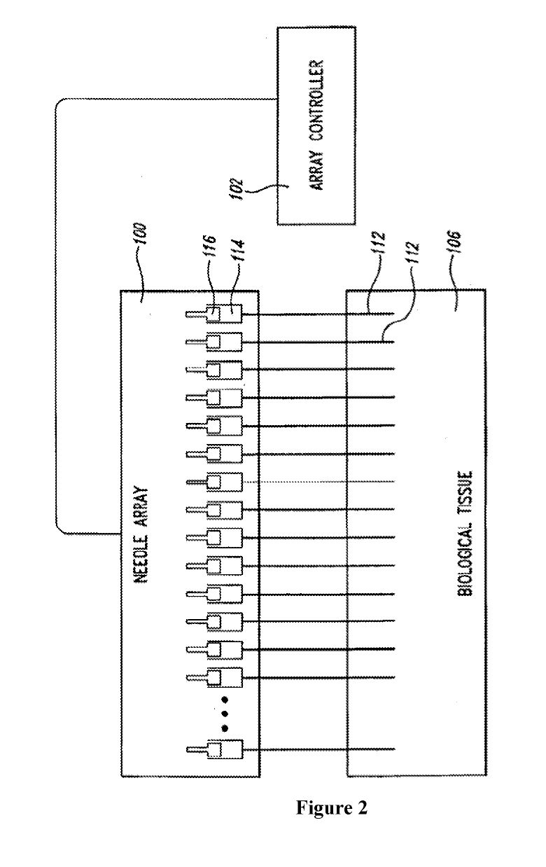

Referring to FIG. 2, a needle array assembly 100 is shown, including a plurality of needles 112, a plurality of reservoirs containing porous tubes 114, a plurality of delivery actuators such as, in the present example, plungers 116, and a controller 102. Each of the plurality of needles 112 is fixed in position relative to the others of the plurality of needles, and the plungers are likewise operatively coupled so as to be fixed in position and simultaneously actuable. Each of the plurality of needles 112 is in fluid communication with a respective one of the plurality of reservoirs 114, and each of the plurality of plungers includes a first end positioned in a respective one of the plurality of reservoirs 114. The controller 102 is operatively coupled to second ends of each of the plurality of plungers 116. The controller is configured to control actuation of the plungers within the reservoir with respect to speed, distance, and direction of movement.

Each of the porous tubes within a reservoir 114 can be charged with a different agent, or some or all of the porous tubes can be charged with a common agent. Movement of the plurality of plungers 116 in a second direction creates a positive pressure, or overpressure, in the respective reservoirs 114, forcing the contents of the reservoirs out via the respective needles 112.

In this configuration, a relatively small amount of a plurality of therapeutic agents can be simultaneously inserted directly to a region of solid tissue 106 for evaluation and analysis. Following insertion, a fluid agent within a porous tube is released to the surrounding tissue by passive diffusion. In some embodiments, the amount of a therapeutic agent delivered to the tissue is less than 1 μL per needle. The evaluation of the tissue 106 and the efficacy of the different therapeutic agents delivered thereto can be used, for example, to screen potential therapeutic agents for subsequent clinical trials or to make subject-specific treatment decisions based on the relative efficacy of the therapeutic agents in the tissue 106.

According to various embodiments, any number of needles can be used. For example, as few as one, two, or three needles can be used, and according to some embodiments, more than one thousand needles can be used. In addition, any types of needle can be used. The needle can be needle with or without pores along its length.

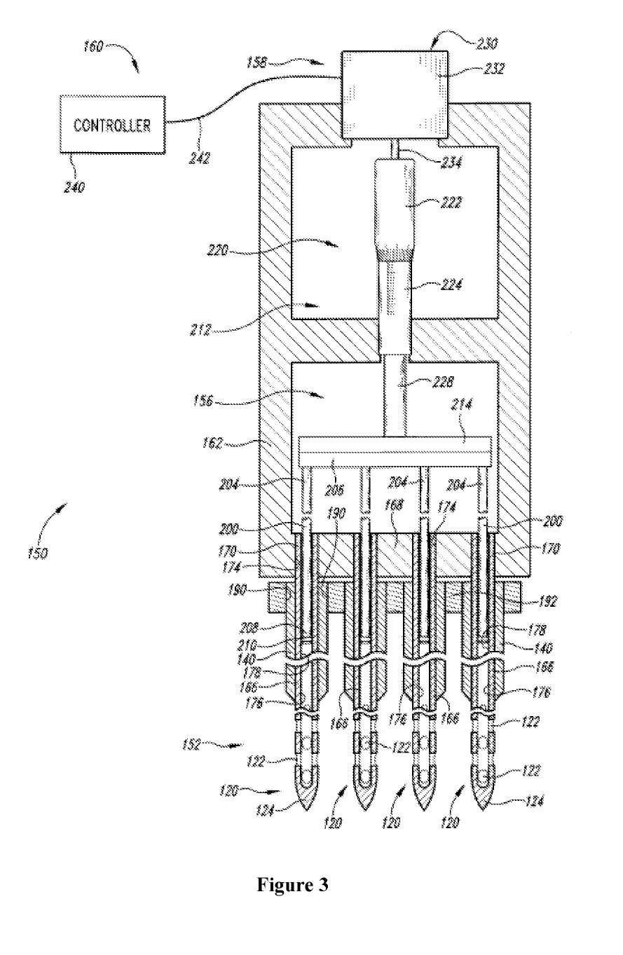

FIG. 3 is a diagrammatic view of a delivery assembly 150 according to another embodiment. The delivery assembly 150 includes a needle array 152, an inserter assembly 190, an actuator assembly 156, a driver assembly 158, a control assembly 240, and a frame 162. The frame 162 provides a substantially rigid structure to which other elements of the assembly 150 are coupled.

The needle array 152 comprises a plurality of needle cylinders 166 and a needle block 168. In the embodiment shown, the needle block 168 is integral with the frame 162. Each of the plurality of needle cylinders 166 is coupled, at a first end 170, in a respective needle aperture 174 extending in the needle block 168, and comprises a lumen 176, having, in the illustrated embodiment, a nominal diameter of 0.15 mm, extending substantially the entire length of the needle cylinder 166. Each needle cylinder 166 includes a reservoir 178 in a region toward the first end 170, a needle 120 in a region toward a second end, and a tip-end 124 at the second end of the needle cylinder 166. In the embodiment shown, the tip-end 124 is tapered to a point.

Each delivery needle 120 is defined by a plurality of ports 122 distributed along its length. The length of each of the plurality of needle cylinders 166 and of the respective needles 120 varies according to the embodiment. In one embodiment, each needle cylinder 166 is longer than 15 cm, while according to other embodiments the needle cylinders are each longer than 10 cm, between 5 cm and 10 cm, and preferably greater than 2 cm, respectively. Likewise, according to various embodiments, each of the plurality of delivery needles 120, defined by the portion of the respective needle cylinder 166 along which the ports 122 are spaced, is longer than 0.1 cm, longer than 2 cm, longer than 4 cm, and longer than 8 cm.

The inserter assembly 190 comprises a plurality of inserter needles 140 coupled to an inserter block 192 in respective inserter apertures 190 extending therein in a configuration that corresponds to the arrangement of the needle cylinders 166 in the needle block 168, such that each of the plurality of needle cylinders 166 can be positioned within a respective one of the plurality of inserter needles 140 as shown in FIG. 3. The inserter assembly 190 is axially slidable over the needle cylinders 166 between a first position, in which only the tip-ends 124 of each of the needle cylinders 166 extend from respective ones of the plurality of inserter needles 140, to a second position, in which the second ends of each of the needle cylinders 166 extends from the respective inserter needle 140 a distance sufficient to clear all of the ports 122 of the respective delivery needle 120.

According to an embodiment, a spacer is provided, configured to be positioned between the inserter block 192 and the needle block 168, sized such that when the inserter block and the needle block are both engaged with the spacer, the inserter block is maintained in the first position. Removal of the spacer permits movement of the inserter block 192 and the needle block 168 relative to each other, to permit placement of the inserter block into the second position, relative to the needle block.

The actuator assembly 156 comprises a plurality of plungers 200 coupled at respective first ends 204 to a plunger block 206 in a configuration that corresponds to the arrangement of the needle cylinders 166 and the inserter needles 140 such that a second end 208 of each of the plurality of plungers 200 can be positioned within the reservoir 178 of a respective one of the plurality of the needle cylinders 166 as shown. An O-ring 210 is provided at the second end 208 of each of the plurality of plungers 200 to sealingly engage the wall of the respective lumen 176. The actuator assembly 156 also comprises an actuator 212 coupled to an actuator block 214, which in turn is rigidly coupled to the plunger block 206. In the embodiment shown, the actuator 212 comprises a micrometer device 220 having a thimble 222, a barrel 224, and a spindle 228 such as are well known in the art. The barrel 224 is rigidly coupled to the frame 162 while the spindle 228 is rotatably coupled to the actuator block 214 so as to control translational movement of the actuator block relative to the frame 162. The micrometer device is calibrated in 0.01 mm increments, with a spindle travel of 0.5 mm per rotation of the thimble 222 and a maximum stroke of 15 mm. Thus, each complete rotation of the thimble moves each of the plurality of plungers 0.5 mm within the lumen 178 of the respective needle cylinder 166 and displaces about 0.0001 cm3 of volume, or 0.1 nL per revolution. Thus, given a maximum stroke of 15 mm, the maximum dispensing capacity of each of the plurality of needles 120 is about 3 nL.

The driver assembly 158 comprises a stepper motor 230 such as is well known in the art, and that includes a motor casing 232, a motor shaft 234 coupled to a rotor of the motor 230, and other elements such as are well known in the art. The motor casing 232 is rigidly coupled to the frame 162, and the motor shaft 234 is slidably coupled to the thimble 222 of the micrometer device while being rotationally locked therewith, such as via a spline coupling, for example. Accordingly, rotational force from the motor shaft 234 is transmitted to the thimble 222, while axial movement of the thimble is not limited by the motor shaft. Such couplings are well known in the mechanical arts. The stepper motor 230 of the illustrated embodiment is configured to divide each rotation into 125 steps. Thus, each incremental rotational step of the motor 230 rotates the thimble about 3°, displacing a volume of about 0.8 pL per reservoir 178.

The controller assembly 160 includes a controller 240 and a control cable 242 that extends from the controller to the stepper motor 230.

Signals for controlling direction, speed, and degree of rotation of the motor shaft 234 are transmitted from the controller 240 to the stepper motor 230 via the control cable 242 in a manner that is well known in the field to which such motors belong. According to an embodiment, the controller is programmable. A user can program the controller to control a speed of delivery from the delivery needles 120 by selecting the speed of rotation, and in some cases a volume of porous tube delivered by selecting the number of partial and complete rotations of the rotor. According to another embodiment, the controller is manually operated, such that a user controls a rate and direction of rotation of the motor 230 in real time. According to a third embodiment, the driver and controller assemblies are omitted, and a user controls delivery by manually rotating the thimble 222 of the actuator assembly 212.

Charging the reservoirs 178 can be accomplished in a number of ways. For example, a charging vessel can be provided that includes a plurality of cups or compartments in an arrangement that corresponds to the arrangement of the needle cylinders 166. The user first places a selected fluidic agent or combination of agents within a porous tube material in each of the cups. The delivery assembly 150 of FIG. 3 is positioned with the needle cylinders pointing downward as shown in the drawing, and the spindle 228 of the actuator 212 fully extended. The frame 162 is lowered until the needles 120 are fully immersed in the contents of the respective cups. The motor 230 is then controlled to rotate in the reverse direction, drawing the spindle 228 inward and pulling the plungers 200 upward. This in turn creates a negative pressure in the reservoirs 178 relative to ambient, drawing the contents into the needle cylinders 166 via the needle ports 122. When the reservoirs are sufficiently charged, rotation of the rotor is halted and the needle array 152 is withdrawn from the charging vessel.

In order to deliver the charge, according to one embodiment, each of the needle cylinders 166 of the needle array 152 is positioned in a respective one of the inserter needles 140 of the inserter assembly 190 so that the tip-ends 178 of the needles 120 protrude from the inserter needles 140. The delivery assembly 190 is then positioned in axial alignment with a target tissue region of a subject and translated axially so that the tip-ends of the needles 120 penetrate the subject's skin. Axial translation of the delivery assembly 190 continues until the tip-ends 124 of the needle cylinders 166 have penetrated to within a selected distance of the target tissue region. The inserter assembly 190 is then held in position while the frame 162 and the elements coupled thereto continue to move axially, such that the needles 120 extend into the target tissue region.

When the needles 120 are correctly positioned, movement of the delivery assembly 190 is halted and the frame 162 is held in position relative to the subject. The stepper motor 230 is then controlled to rotate the thimble 222 in the forward direction so as to cause the spindle 228 to extend, driving the plungers 200 into the needle cylinders 166 and creating an overpressure in the respective reservoirs 178, thereby forcing contents from the reservoirs to the target tissue region via the ports 122 of the delivery needles 120.

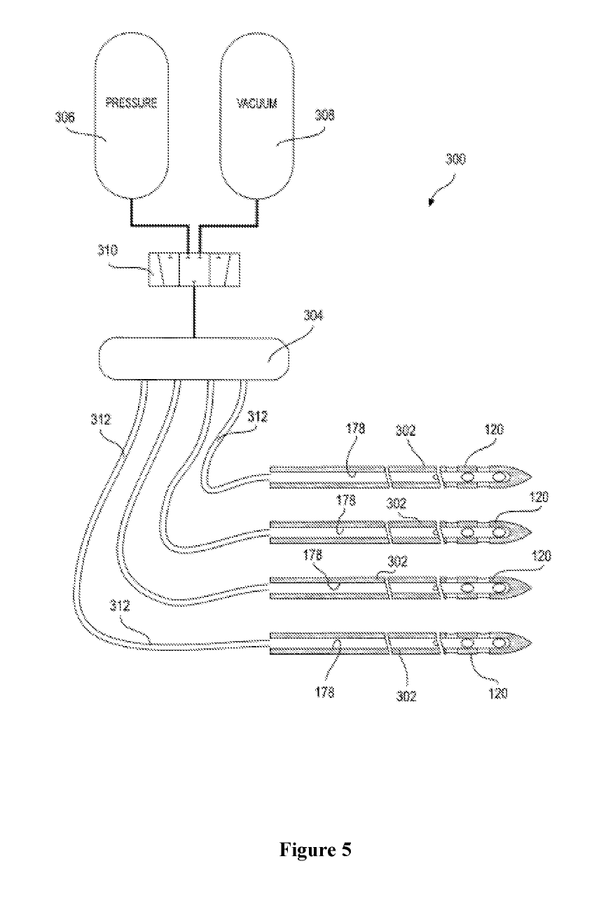

Delivery can be performed in a few seconds, or it can be extended over minutes or hours under a relatively low overpressure to promote complete absorption of the reservoir contents into the surrounding tissue. According to the embodiment described with reference to FIG. 5, the stepper motor 230 can be controlled to rotate the rotor fast enough to depress the plungers 200 the full 15 mm in less than one second, or slow enough that a single rotation can take many hours.

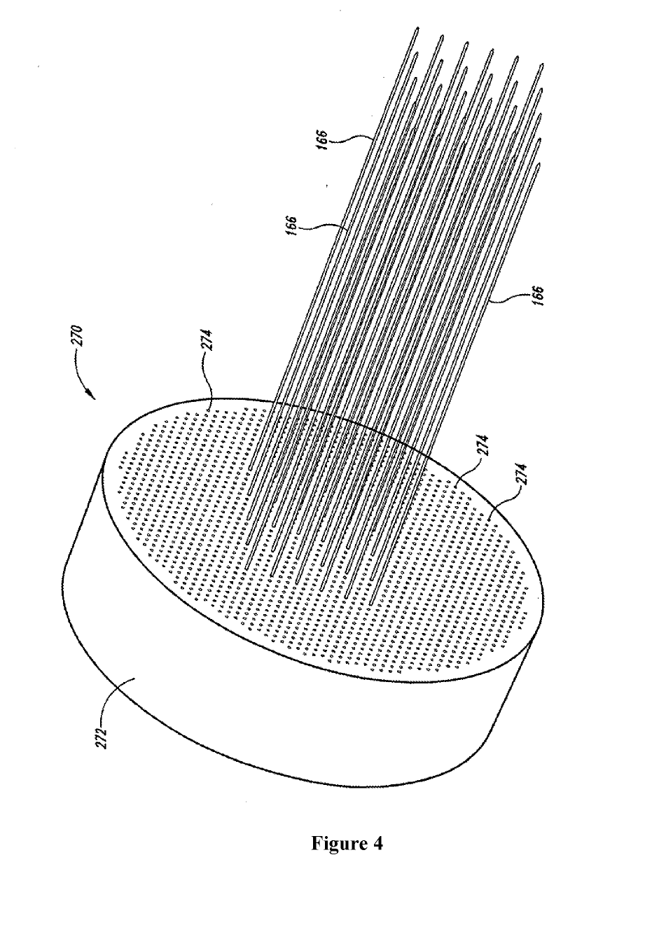

Turning now to FIG. 4, elements of a delivery assembly 270 are shown according to another embodiment. A needle block 272 includes a large plurality of needle apertures 274 extending therethrough, arranged in a closely spaced array. Needle cylinders 166 are provided separately, in various assortments of lengths and numbers, sizes, and spacings of ports.

In use, a user selects a number of needles to be used for a particular procedure, and selects the particular needle cylinders 166, placing each in a respective one of the plurality of apertures 274 of the needle block, in an arrangement that is selected for the particular procedure. The user can require only a small number of needles; such as one to five, for example, or can require hundreds or thousands of needles. Furthermore, the needle cylinders 166 can be of varying lengths and configurations. The needles may be pre-loaded with fluid agents within porous tubes. The user selects the arrangement of the needle cylinders 166 in the needle block 272, and their respective lengths and configurations, at least in part according to factors such as the size, shape, and position of a target tissue region in a subject's body, the desired distribution density of fluid in the target tissue region, the permeability of the target tissue, etc.

The delivery actuators of previous embodiments have been described as plungers. However, any suitable actuator can be used to control an amount of therapeutic agent delivered from the reservoirs into the needle. For example, fluid pressure such as by compressed air or pressurized liquid can be used to control an amount of therapeutic agent delivered to a region of biological tissue via the porous tubes and needles.

Referring now to FIG. 5, a delivery assembly 300 is shown, according to another embodiment. The delivery assembly 300 includes a plurality of needle cylinders 302 comprising respective reservoirs 178 and needles 120. Fluid couplings 312 place the needle cylinders 302 in fluid communication with a manifold 304. A fluid pressure source 306 and a fluid vacuum source 308 can each be placed in fluid communication with the manifold 304 by operation of a valve 310.

According to the embodiment of FIG. 5, the needle cylinders 302 are not fixed with respect to each other, but can be individually emplaced, in a target tissue region, for example. The reservoirs may be charged by placing the delivery needles 120 in a selected fluid, e.g., a therapeutic agent or respective therapeutic agent, and the fluid vacuum source is placed in fluid communication with the manifold, drawing a negative pressure into the reservoirs and drawing the agent into the needles. The user then positions the needles 120 in the target tissue region. When they are all in place, the manifold 304 is pressurized, forcing fluid from the reservoirs of each of the needle cylinders 302 via the ports of the respective delivery needles. While FIG. 5 shows a simple fluid circuit, it will be understood that in practice such a circuit could include any of valves, pressure regulator, peristaltic pump, microfluidic pump, vacuum accumulator, compressor, controller, etc., all of which are well known in the art, and within the abilities of one of ordinary skill to select and configure for a given application.

### Screening Candidate Agents

A drug-delivery device comprising porous tubes of the invention is useful for methods of administering candidate therapeutic agents to a subject by depositing one or more porous tubes packed with one or more candidate agents in the a tissue of the subject. Spatially constrained delivery of a plurality of candidate agents permits parallel evaluation of agents for effect on a tissue such as a tumor.

A fluid agent within a porous tube may contain a dye, useful in monitoring the response to a candidate agent. A dye may be a position marker, or it may be chosen to report, for example, by fluorescence, the activation or inactivation of a biological function upon imaging. This process allows an experimenter to make a direct assessment of the affect of a candidate agent on a physiological system of interest.

Detection of an effect of a candidate agent can be performed by assessing an alteration in a biological function of a cell or tissue. In some embodiments, the biological function is a pathway, an activity of an enzyme, an expression of a gene, transcription of the gene, translation of an RNA molecule associated with the gene, or peptide synthesis. In some embodiments, the biological function is associated with cancer, degenerative disease, inflammation, metabolism, apoptosis, or an immune response. In some embodiments, the biological function is associated with cancer. In some embodiments, the pathway is a cancer pathway. In some embodiments, the enzyme is associated with cancer. In some embodiments, the gene is associated with cancer. In some embodiments, the pathway is an apoptotic pathway, and the dye reports apoptosis.

In some embodiments, the gene is ABL1, ABL2, ACSL3, AF15Q14, AF1Q, AF3p21, AF5q31, AKAP9, AKT1, AKT2, ALK, ALO17, APC, ARHGEF12, ARHH, ARNT, ASPSCR1, ASXL1, ATF1, ATIC, ATM, BCL10, BCL11A, BCL11B, BCL2, BCL3, BCL5, BCL6, BCL7A, BCL9, BCR, BHD, BIRC3, BLM, BMPR1A, BRAF, BRCA1, BRCA2, BRD3, BRD4, BRIP1, BTG1, BUB1B, C12orf9, C15orf21, CANT1, CARD11, CARS, CBFA2T1, CBFA2T3, CBFB, CBL, CBLB, CBLC, CCND1, CCND2, CCND3, CD74, CD79A, CD79B, CDH1, CDH11, CDK4, CDK6, CDKN2A-p14ARF, CDKN2A-p16(1NK4a), CDKN2C, CDX2, CEBPA, CEP1, CHCHD7, CHEK2, CHIC2, CHN1, CIC, CLTC, CLTCL1, CMKOR1, COL1A1, COPEB, COX6C, CREB1, CREB3L2, CREBBP, CRLF2, CRTC3, CTNNB1, CYLD, D10S170, DDB2, DDIT3, DDX10, DDX5, DDX6, DEK, DICER1, DUX4, EGFR, EIF4A2, ELF4, ELK4, ELKS, ELL, ELN, EML4, EP300, EPS15, ERBB2, ERCC2, ERCC3, ERCC4, ERCC5, ERG, ETV1, ETV4, ETV5, ETV6, EVI1, EWSR1, EXT1, EXT2, EZH2, FACL6, FANCA, FANCC, FANCD2, FANCE, FANCF, FANCG, FBXW7, FCGR2B, FEV, FGFR1, FGFR10P, FGFR2, FGFR3, FH, FIP1L1, FLI1, FLT3, FNBP1, FOXL2, FOXO1A, FOXO3A, FOXP1, FSTL3, FUS, FVT1, GAS7, GATA1, GATA2, GATA3, GMPS, GNAQ, GNAS, GOLGA5, GOPC, GPC3, GPHN, GRAF, HCMOGT-1, HEAB, HEI10, HERPUD1, HIP1, HIST1H4I, HLF, HLXB9, HMGA1, HMGA2, HNRNPA2B1, HOOKS, HOXA11, HOXA13, HOXA9, HOXC11, HOXC13, HOXD11, HOXD13, HRAS, HRPT2, HSPCA, HSPCB, IDH1, IDH2, IGH@, IGK@, IGL@, IKZF1, IL2, IL21R, IL6ST, IRF4, IRTA1, ITK, JAK1, JAK2, JAK3, JAZF1, JUN, KDM5A, KDM5C, KDM6A, KDR, KIAA1549, KIT, KLK2, KRAS, KTN1, LAF4, LASP1, LCK, LCP1, LCX, LHFP, LIFR, LMO1, LMO2, LPP, LYL1, MADH4, MAF, MAFB, MALT1, MAML2, MAP2K4, MDM2, MDM4, MDS1, MDS2, MECT1, MEN1, MET, MHC2TA, MITF, MKL1, MLF1, MLH1, MLL, MLLT1, MLLT10, MLLT2, MLLT3, MLLT4, MLLT6, MLLT7, MN1, MPL, MSF, MSH2, MSH6, MSI2, MSN, MTCP1, MUC1, MUTYH, MYB, MYC, MYCL1, MYCN, MYH11, MYH9, MYST4, NACA, NBS1, NCOA1, NCOA2, NCOA4, NF1, NF2, NFIB, NFKB2, N1N, NONO, NOTCH1, NOTCH2, NPM1, NR4A3, NRAS, NSD1, NTRK1, NTRK3, NUMA1, NUP214, NUP98, NUT, OLIG2, OMD, P2RY8, PAFAH1B2, PALB2, PAX3, PAX5, PAX7, PAX8, PBX1, PCM1, PCSK7, PDE4DIP, PDGFB, PDGFRA, PDGFRB, PER1, PHOX2B, PICALM, PIK3CA, PIK3R1, PIM1, PLAG1, PML, PMS1, PMS2, PMX1, PNUTL1, POU2AF1, POU5F1, PPARG, PRCC, PRDM16, PRF1, PRKAR1A, PRO1073, PSIP2, PTCH, PTEN, PTPN11, RAB5EP, RAD51L1, RAF1, RANBP17, RAP1GDS1, RARA, RB1, RBM15, RECQL4, REL, RET, ROS1, RPL22, RPN1, RUNX1, RUNXBP2, SBDS, SDH5, SDHB, SDHC, SDHD, SEPT6, SET, SETD2, SFPQ, SFRS3, SH3GL1, SIL, SLC45A3, SMARCA4, SMARCB1, SMO, SOCS1, SRGAP3, SS18, SS18L1, SSH3BP1, SSX1, SSX2, SSX4, STK11, STL, SUFU, SUZ12, SYK, TAF15, TAL1, TAL2, TCEA1, TCF1, TCF12, TCF3, TCL1A, TCL6, TET2, TFE3, TFEB, TFG, TFPT, TFRC, THRAP3, TIF1, TLX1, TLX3 TMPRSS2, TNFAIP3, TNFRSF17, TNFRSF6, TOP1, TP53, TPM3, TPM4, TPR, TRA@, TRB@, TRD@, TRIM27, TRIM33, TRIP11, TSC1, TSC2, TSHR, TTL, USP6, VHL, WAS, WHSC1, WHSC1L1, WRN, WT1, WTX, XPA, XPC, ZNF145, ZNF198, ZNF278, ZNF331, ZNF384, ZNF521, ZNF9, mTOR, MEK, PI3K, HIF, IGF1R, GLS1, or ZNFN1A1. In some embodiments, the pathway is associated with any of the aforementioned genes. In some embodiments, the peptide of the peptide synthesis is a gene product of any of the aforementioned genes.

In some embodiments, the target tissue does not exhibit features of a disease, and a dye may be used to assess the response of an individual tissue to one or more compounds. In some cases, one or more compounds may be administered to produce an altered physiologic state within a tissue. An altered physiologic state can be any detectable parameter that directly relates to a condition, process, pathway, dynamic structure, state or other activity in a solid tissue (and in some embodiments in a solid tumor) including in a region or a biological sample that permits detection of an altered (e.g., measurably changed in a statistically significant manner relative to an appropriate control) structure or function in a biological sample from a subject or biological source. The methods of the present invention thus pertain in part to such correlation where an indicator of altered physiologic state can be, for example, a cellular or biochemical activity, including as further non-limiting examples, cell viability, cell proliferation, apoptosis, cellular resistance to anti-growth signals, cell motility, cellular expression or elaboration of connective tissue-degrading enzymes, cellular recruitment of angiogenesis, or other criteria as provided herein.

Altered physiologic state can further refer to any condition or function where any structure or activity that is directly or indirectly related to a solid tissue function has been changed in a statistically significant manner relative to a control or standard, and can have its origin in direct or indirect interactions between a solid tissue constituent and an introduced agent, or in structural or functional changes that occur as the result of interactions between intermediates that can be formed as the result of such interactions, including metabolites, catabolites, substrates, precursors, cofactors and the like. Additionally, altered physiologic state can include altered signal transduction, respiratory, metabolic, genetic, biosynthetic or other biochemical or biophysical activity in some or all cells or tissues of a subject or biological source, in some embodiments in some or all cells of a solid tissue, and in some embodiments in some or all cells of a tumor such as a solid tumor in a solid tissue. As non-limiting examples, altered biological signal transduction, cell viability, cell proliferation, apoptosis, cellular resistance to anti-growth signals, cell motility, cellular expression or elaboration of connective tissue-degrading enzymes, cellular recruitment of angiogenesis, or other criteria including induction of apoptotic pathways and formation of atypical chemical and biochemical crosslinked species within a cell, whether by enzymatic or non-enzymatic mechanisms, can all be regarded as indicative of altered physiologic state.

According to an embodiment, a solid tissue into which a plurality of therapeutic agents has been delivered is subsequently excised from the subject and evaluated. For example, in a case where the target tissue is a cancerous tumor, the plurality of agents injected therein can include some agents whose efficacy or effect on such tumors is under investigation. By injecting the various agents in vivo then waiting a selected period before removing the tumor, the effect of the agents on the tumor in situ can be investigated. This preserves the tumor microenvironment and distinguishes this method from current ex vivo or in vitro therapeutics evaluation methods. Over time, each agent permeates outward from its delivery axis to a greater or lesser degree, depending on factors such as, for example, the density of the surrounding tissue, the viscosity and composition of the agent, the wettability of the tissue by the respective agent, etc. Typically, the portions of the tissue into which the agents spread are approximately column-shaped regions coaxial with the respective delivery axes.

According to various embodiments, a region of tissue is left in place for some period of time before being excised. For example, a period of 48-72 hours following delivery is thought to be generally sufficient for a tumor to exhibit a detectable response. In other cases, the wait period can be minutes, hours, days, or weeks. According to some embodiments, the tissue region is imaged using known methods to precisely locate the target region of tissue prior to insertion of the needles. The region can be imaged repeatedly before and after delivery of the plurality of agents to the region of tissue. In some embodiments, the evaluation is carried out on at least one portion of the tissue that has previously been excised.

In some embodiments, the excised tissue can be cut into a plurality of serial histological sections along parallel planes that are substantially normal (e.g., perpendicular or deviating from perpendicular by as much as 1, 2, 3, 4, 5, 6, 7, 8, 9, 10, 11, 12, 13, 14, 15, 20, 25, 30, 35 or more degrees) to the parallel axes, for analysis by any of a number of known histological, histochemical, immunohistological, histopathologic, microscopic (including morphometric analysis and/or three-dimensional reconstruction), cytological, biochemical, pharmacological, molecular biological, immunochemical, imaging or other analytical techniques, which techniques are known to persons skilled in the relevant art. See, e.g., Bancroft and Gamble, Theory and Practice of Histological Techniques (6^{th} Ed.) 2007 Churchill Livingstone, Oxford, UK; Kieman, Histological and Histochemical Methods: Theory and Practice, 2001 Cold Spring Harbor Laboratory Press, Cold Spring Harbor, N.Y.; and M. A. Hayat (Ed.), Cancer Imaging—Vols. 1 and 2, 2007 Academic Press, NY, each of which is incorporated by reference herein in its entirety. Imaging can be performed before, during or after dispenser needles are inserted into the solid tissue.

According to other embodiments, a plurality of agents is delivered to a portion of tissue via respective ones of a plurality of needles of a needle array after the portion of tissue is excised.

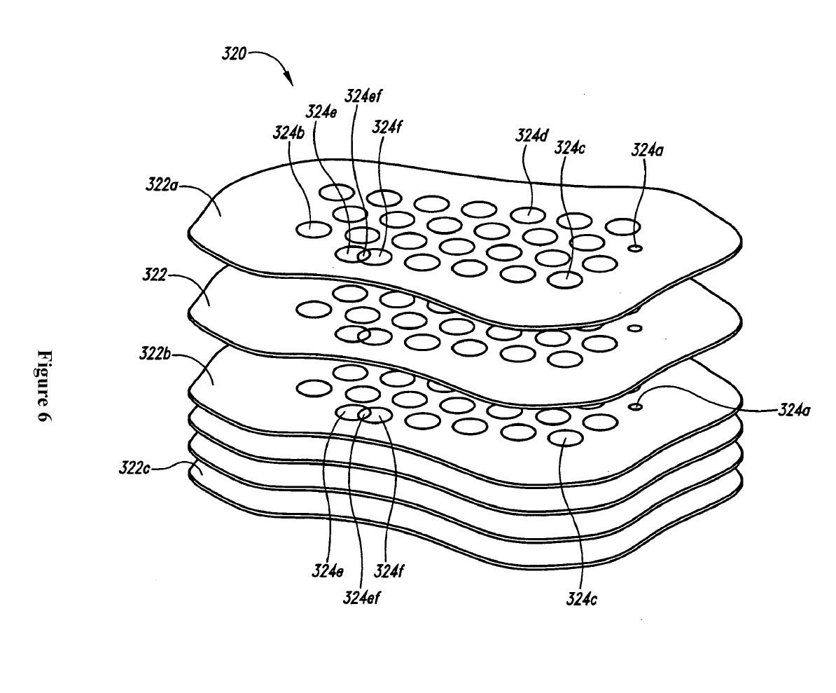

Referring now to FIG. 6, a portion of a tumor 320 is shown, following an injection procedure and subsequent resection. The tumor 320 has been sectioned into a plurality of slices 322 along planes that lie substantially normal to the delivery axes. Column-shaped delivery regions 324 define the regions of permeation of the respective agents, and extend perpendicular to the planes of the sections 322

Many of the regions 324 may not be easily detectable to a user, so generally at least two readily detectable position markers 324a, 324b are among the agents injected, at widely separated locations. The user can then overlay a template on which the locations of each of the delivery axes is marked, aligning the indicated marker positions of the template with the detectable position markers 324a, 324b of a given section 322, thereby locating the remaining delivery regions 324. The position markers 324a, 324b can be any composition that is detectable by a user. Various exemplary position markers are described in detail elsewhere in this disclosure. According to an embodiment, the position markers are selected to resist permeation and diffusion into the surrounding tissue and to remain concentrated in a narrow column, as shown for example at 324a, so as to be detectable for an extended period after the injection procedure, and to provide an accurate guide for positioning the template.

In addition to position markers, control agents can also be among the agents injected. For example, a negative control can comprise a substance used as a vehicle in others of the agents, and a positive control can comprise a compound of most or all of the agents delivered individually at other delivery axes.

Following sectioning of the tumor 320, a user conducts selected assays on delivery regions 324 of various sections 322 of the tumor 320, as described in more detail later. One benefit of the devices and methods disclosed herein is that, in addition to evaluating the efficacy of a given agent on the tumor, the efficacy of agents at various delivery regions 324 can be evaluated and compared. Additionally, the effect of a given agent on various parts of the tumor can be evaluated, both vertically and horizontally. By comparing the effect of an agent in a delivery region 324c at section 322a, for example, with its effect in the same region 324c at sections 322b and 322c, the effect of that agent on different tissue compositions that can occur vertically can be differentiated. Similarly, the same agent can be delivered at several delivery axes in the array, e.g., 324c and 324d, and the relative effects at those locations in a given section 322 can then be compared, providing horizontal differentiation. As is well known in the art, biological tissue is rarely homogeneous over even relatively small distances. A given agent might have substantially no effect on some tissue structures of a tumor, but might, on the other hand, be extremely effective on others. Such differential effects can be detected and evaluated as described above.

Another valuable aspect that can be evaluated is the effect of multiple agents in regions where they interact within the tissue. Delivery regions 324e and 324f are spaced more closely together than the others, resulting in the respective agents interacting in a region 324ef where the respective delivery regions overlap.

According to certain presently contemplated embodiments, the efficacy of a candidate agent can be identified by detecting an altered physiologic state as provided herein, including by assessing any of a number of biological parameters characteristic of a cancer cell such as those reviewed by Hanahan and Weinberg (2000 Cell 100:57) and in the references cited therein. Therein are disclosed methodologies for determining the effect of a candidate agent on one or more traits exhibited by cancer cells, and detectable by any of a variety of techniques known to the art for determining one or more of (i) an ability to evade apoptosis, (ii) acquisition of self-sufficiency in growth signals, (iii) insensitivity to growth-inhibitory signals, (iv) acquisition of tissue invasive and metastatic phenotype, (v) unlimited replicative potential, and (vi) sustained angiogenesis. Persons skilled in the art are familiar with multiple approaches for detecting the presence of these alterations of physiologic state, which can be adapted to a particular excised tumor system. See, e.g., Bonificano et al. (Eds.) Current Protocols in Cell Biology, 2007 John Wiley & Sons, NY; Ausubel et al. (Eds.) Current Protocols in Molecular Biology, 2007 John Wiley & Sons, NY; Coligan et al. (Eds.), Current Protocols in Immunology, 2007 John Wiley & Sons, NY; Robinson et al. (Eds), Current Protocols in Cytometry, 2007 John Wiley & Sons, NY. Non-limiting examples of parameters that can be assayed to identify an altered physiologic state include assays of cell viability, cell division, apoptosis, necrosis, cell surface marker expression, cellular activation state, cellular elaboration of extracellular matrix (ECM) components or of ECM-degrading enzymes, morphometric analysis, extension or retraction of cellular processes, cytoskeletal reorganization, altered gene expression, e.g., by in situ hybridization of immunohistochemistry (e.g., Shibata et al., 2002 J. Anat. 200:309) intracellular phosphoprotein localization (e.g., Gavet et al., 1998 J Cell Sci 111:3333), and the like.

As described herein, determination of levels of at least one indicator of altered physiologic state can also be used to stratify a subject population for eligibility to participate in a clinical trial. These and related embodiments are contemplated as usefully providing advantages associated with evaluation of candidate therapeutic compounds at an earlier stage of development than is currently the case. For instance, it is not currently standard clinical trial practice to establish biomarker parameters (which can be the basis for exclusion of subjects) prior to Phase Ill studies, whereas the embodiments described herein can provide useful results even in the absence of established biomarker criteria, for example, at Phase II. Accordingly it is envisioned that through the practice of certain presently disclosed embodiments, relevant information on the properties of a candidate agent can be obtained earlier in a solid tumor oncology drug development program than has previously been the case, including in a manner which can time-efficiently and cost-effectively permit elimination from a clinical trial of subjects for whom no response or benefit can be expected based on a nonresponder result for a particular candidate agent.

For example, stratification of a subject population according to levels of at least one indicator of altered physiologic state, determined as described herein, can provide a useful marker with which to correlate the efficacy of any candidate therapeutic agent being used in cancer subjects, and/or to classify subjects as responders, nonresponders or possible responders.

In some embodiments, the method is useful in drug screening and drug discovery, such as in preclinical animal models to identify and functionally characterize potential new therapeutics. For instance, a plurality of siRNAs can be administered intratumorally and their relative abilities to knock down expression of a desired target gene can be compared. Other similar embodiments can find uses in clinical contexts, for example, to “deselect”, or eliminate from consideration, known therapeutic agents that have no effect in a particular tumor, thereby advantageously advancing the therapeutic management of a subject by avoiding the loss of time and the undesirable side-effects that can be associated with administering an ineffectual treatment regimen.

Some embodiments include those in which the solid tissue comprises a tumor, wherein agent delivery can be made to, and/or sample retrieval can be made from, the solid tumor. It will be appreciated by persons familiar with the art from the disclosure herein that in the course of practicing certain embodiments described herein, a selected region of a tumor can comprise the site into which the needles of the presently described devices are inserted, introduced or otherwise contacted with the tumor. The region can be selected on any number of bases, including based on imaging that can be conducted before, during or after a step of needle insertion, introduction or contacting, or based on imaging conducted before, during or after excising the solid tissue from a subject, or based on other criteria including but not limited to anatomic location, accessibility in the course of a surgical procedure, degree of vascularization or other criteria.

### Data Acquisition and Analysis

In some embodiments, it is contemplated that the target region in a solid tissue can be imaged using known techniques to evaluate the effects of the agents. The imaging can be by any suitable process or method, including, for example, radiographic imaging, magnetic resonance imaging, positron emission tomogoraphy, biophotonic imaging, etc. In some embodiments, the target region can be imaged repeatedly before, during, and after the delivery process.

Upon imaging, the level of the reporting signal can be quantified by methods known to one of skill in the art. Observation and/or quantification of the reporting signal can be used to make informed research and health care decisions regarding the use and efficacy of a therapeutic agent. Non-limiting examples of decisions that can be made on such observations include fluid volume quality control, positional tracking, and drug biodistribution. Such experiments can be performed on a lower mammal, for example, a mouse, to provide reporting signals that can be used to make informed predictions regarding the activity of a potential therapeutic agent in a human Animal studies of this type can be used to avoid the inherent uncertainty and inaccuracies that arise by conducting drug efficacy studies in cells in controlled environments instead of in the native environment.

Quantification of fluorescence signals can be accomplished by any method known in the art. Fluorescence signals can be compared with a standard or a control to determine up-regulation or down-regulation of a biological pathway. Such observations can be used to make predictions regarding the therapeutic value of drug candidates.

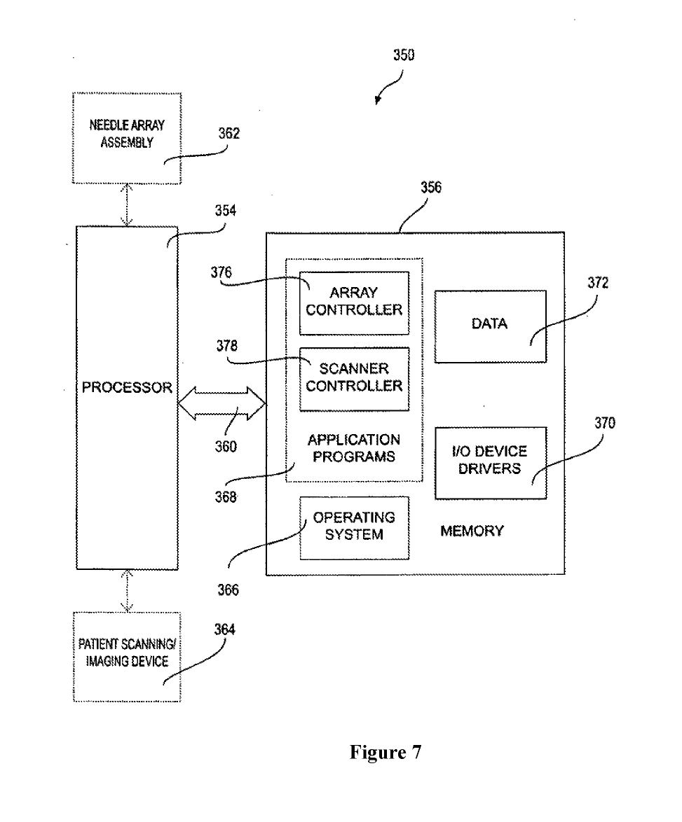

According to FIG. 7, a data processing system 350 is used to carry out or direct operations, and includes a processor 354 and a memory 356. The processor 354 communicates with the memory 356 via an address/data bus 360 and also communicates with a needle array assembly 362 and a subject scanning device 364. The subject scanning device 364 is used, according to an embodiment, to assist in placing the needles of the needle array assembly 362 in a subject in vivo and for non-invasive analysis of target tissue regions using imaging techniques, such as radiographic imaging or nuclear medical assays. The processor 354 can be a commercially available or custom microprocessor, microcontroller, signal processor or the like. The memory 356 can include any memory devices and/or storage media containing the software and data used to implement the functionality circuits and modules.

The memory 356 can any of include several categories of software and data used in the data processing system, such as, for example, an operating system 366, application programs 368; input/output device drivers 370; and data 372. The application programs 368 are illustrative of the programs that implement the various features of the circuits and modules according to some embodiments, and the data 372 represents the static and dynamic data used by the application programs 368, the operating system 366, the input/output device drivers 370 and other software programs that can reside in the memory 356.

According to various embodiments, the data processing system 350 can include several modules, including an array controller 376, a scanner controller 378 and the like. The modules can be configured as a single module or additional modules otherwise configured to implement the operations described herein. For example, the array controller 376 can be configured to control the needle array assembly 100 of FIG. 2, by controlling the actuators 116, and consequently, the release of therapeutic agents from the reservoirs 114 via the needles 112. The scanner controller 378 can be configured to control the subject scanning device 364.

In some embodiments, detection in a solid tissue of an altered physiologic state subsequent to introducing an agent or a plurality of agents includes detecting a degree of permeation of the agent(s) through the solid tissue, detecting a degree of absorption of the agent(s) in the tissue, detecting a physicochemical effect of the agent(s) on the tissue, and/or detecting a pharmacological effect of the agent(s) on the tissue. Assays, including fluorescence assays, of drug permeation or penetration in solid tissues are known in the art and have been described (e.g., Kerr et al., 1987 Canc. Chemother. Pharmacol. 19:1 and references cited therein; Nederman et al., 1981 In Vitro 17:290; Durand, 1981 Canc. Res. 41:3495; Durand, 1989 JNCI 81:146; Tunggal et al., 1999 Clin. Canc. Res. 5:1583) and can be configured further according to the present disclosure, for instance, through the detection in histological serial sections of a detectable label that has been co-administered to the solid tissue, prior to excision and sectioning, with an agent of interest.

In such embodiments, permeation or penetration refers to the area of retention of an agent in the solid tissue in the immediate vicinity of the needle from which the agent was introduced exclusive of perfusion (entry into and dispersion via any blood vessel), and can include retention of the agent in extracellular space or extracellular matrix or in association with a cell membrane or intracellularly. Permeation can be distinct from a physicochemical effect, which refers to microscopically detectable mechanical disruption of tissue that results from the needle insertion or fluid injection itself, or from non-biological mechanical or chemical tissue disruption caused by the agent (e.g., damage to cell membranes or disintegration of cell-cell junctions). Pharmacological effects include statistically significant alterations of a cell or tissue physiological state that are detectable as consequences of the molecular mechanism of action of the agent, for example, cytoskeletal reorganization, extension or withdrawal of cellular processes, or evidence of biological signal transduction as can be detected using any of a number of known cytological, biochemical, molecular biological or other read-outs. Comparison of serial sections can permit distinguishing the nature of the effect that is detected histologically.

### EXAMPLES

### Example 1

### Spatially Restricted Delivery of Dye at Multiple Tumor Depths

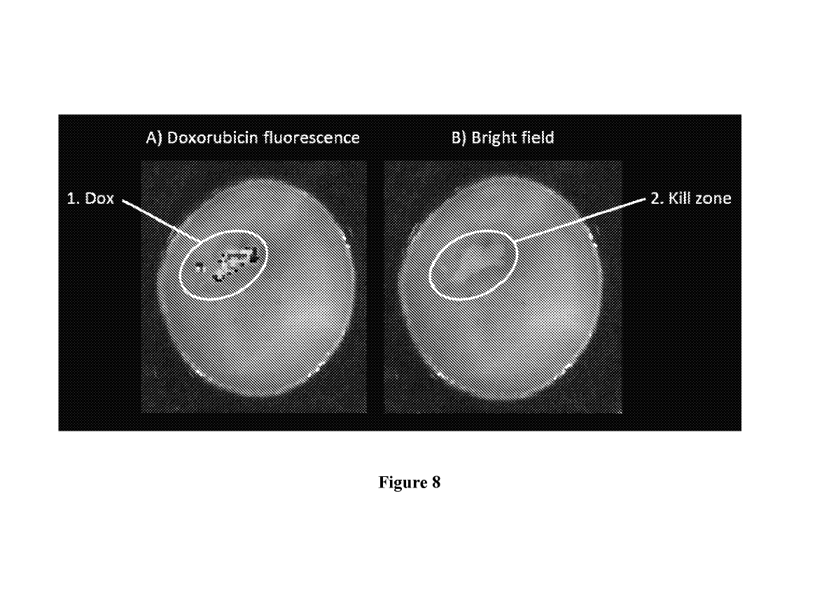

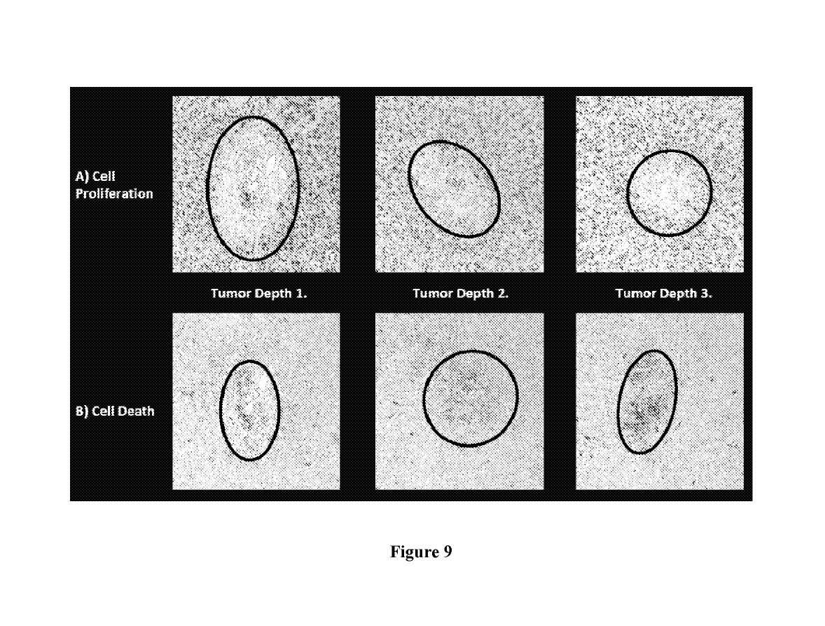

Doxorubicin was delivered to a lymphoma tumor using a needle array of the method. The tumor was then excised and sectioned. FIG. 8 shows a slice of the tumor, imaged using fluorescence and brightfield microscopy. These images show that doxorubicin fluorescence overlaps with the region of dead cells discernible in the brightfield image. Regions of doxorubicin fluorescence and cell death are localized to a zone within the tumor slice, reflecting spatially constrained doxorubicin delivery. FIG. 9 shows three tumor cross-sectional slices from different depths, and demonstrates that the localized delivery depicted in FIG. 8 extends to various tumor depths. Cell death was observed in a localized area across the three tumor depths shown.

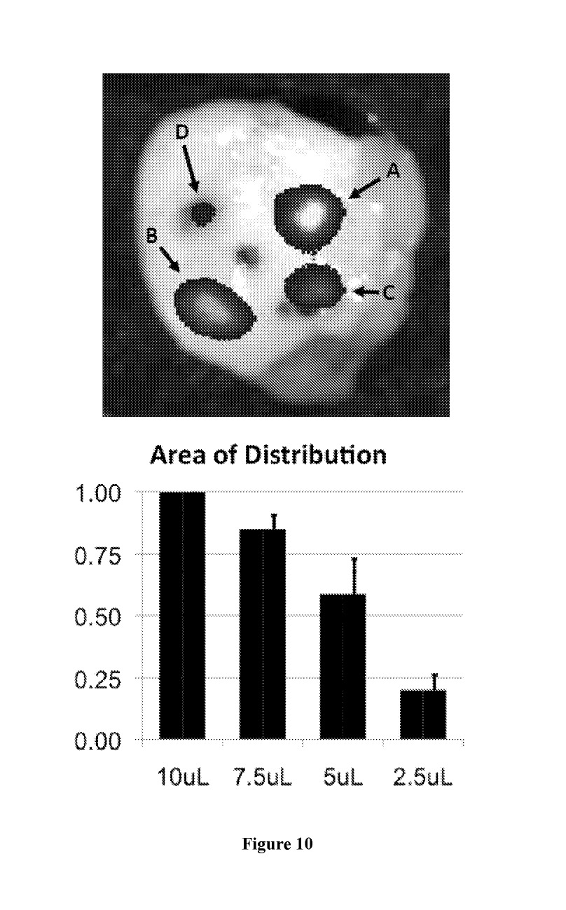

Spatially restricted delivery was tested by injecting four different volumes of a fluorescent dye using a needle array. The dye was injected along four parallel axes within a tumor. FIG. 10 illustrates fluorescent microscopy of these injections, and the resulting spatially-restricted distribution of fluorescent dye (top panel). The injections were A) 10 μL; B) 7.5 μL; C) 5 μL; and D) 2.5 μL. The graph in the bottom panel depicts the relative areas of distribution, averaged over 15 sections from different tumor depths, for each injection.

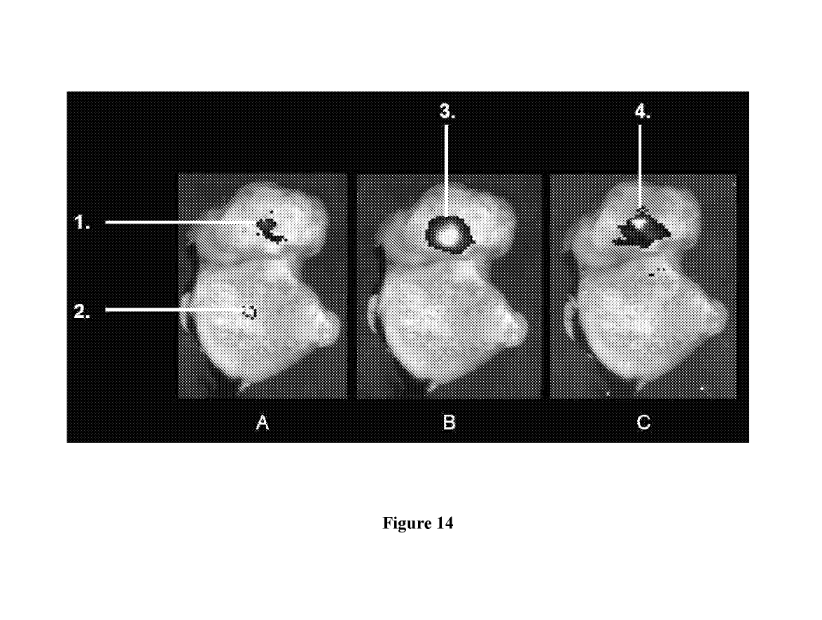

FIG. 14 depicts the use of various indicator dyes according to the method, to monitor spatially restricted delivery of compounds and resulting localized effects on regions of a tumor. A mouse tumor was injected with 1. doxorubicin; and 2. a control. Panel A illustrates excitation at 640 nm and emission at 800 nm of the dye. Panel B illustrates the doxorubicin signal (3.) observed after excitation at 500 nm and emission at 600 nm of doxorubicin. Panel C illustrates an apoptosis signal (4.) observed after excitation at 640 nm and emission at 720 nm of the dye. The results show that apoptosis overlaps with the region that received doxorubicin, but not control, demonstrating spatially restricted drug delivery and tumor cell killing.

### Example 2

### Comparison of In Vivo and In Vitro Analyses

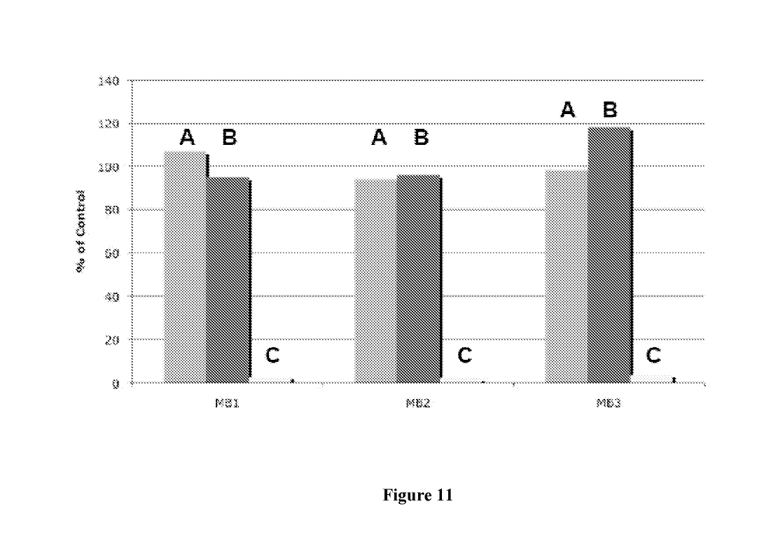

Sonic hedgehog (Shh) antagonists were tested in vitro and in vivo, and the results were compared. FIG. 11 illustrates an in vitro response to hedgehog pathway antagonism in a human medulloblastoma sample. Medulloblastoma cells were taken from three patients and cultured in vitro. Samples from the three patients, MB1-MB3, were tested for the effect of Shh antagonists, which showed no response compared to positive control in this study. Bars A) depicts injection of 1 μM of SHH antagonist; Bars B) injection of 5 μM SHH antagonist; and C) injection of a control.

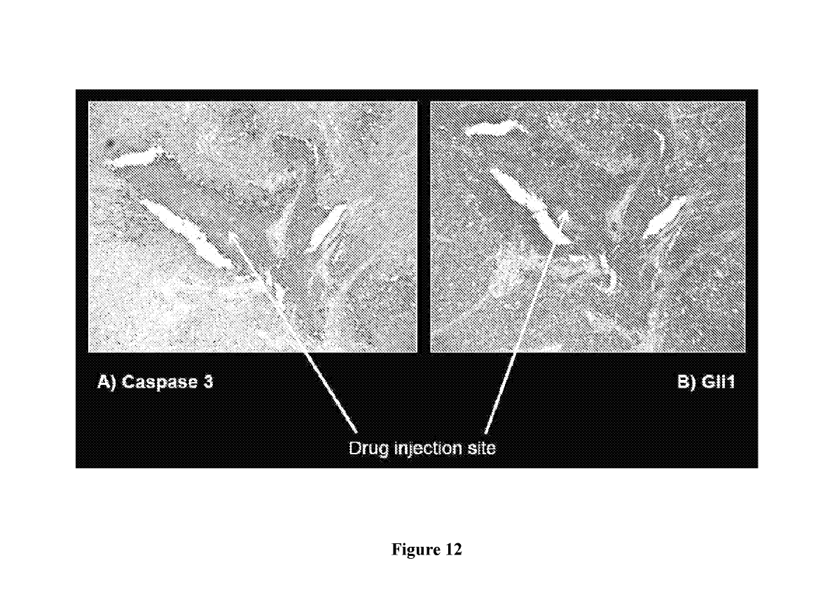

In contrast to the results of the in vitro experiment shown in FIG. 11, FIG. 12 illustrates the response of Shh antagonist injection to a tumor in vivo. Shh antagonists were injected in the tumor in a spatially-restricted fashion, using the method of the invention, and visualized by fluorescent microscopy. FIG. 12 shows brightfield microscopy of localized positive signal for A) caspase 3; and B) Gli1, illustrating spatially restricted tumor kill in both cases.

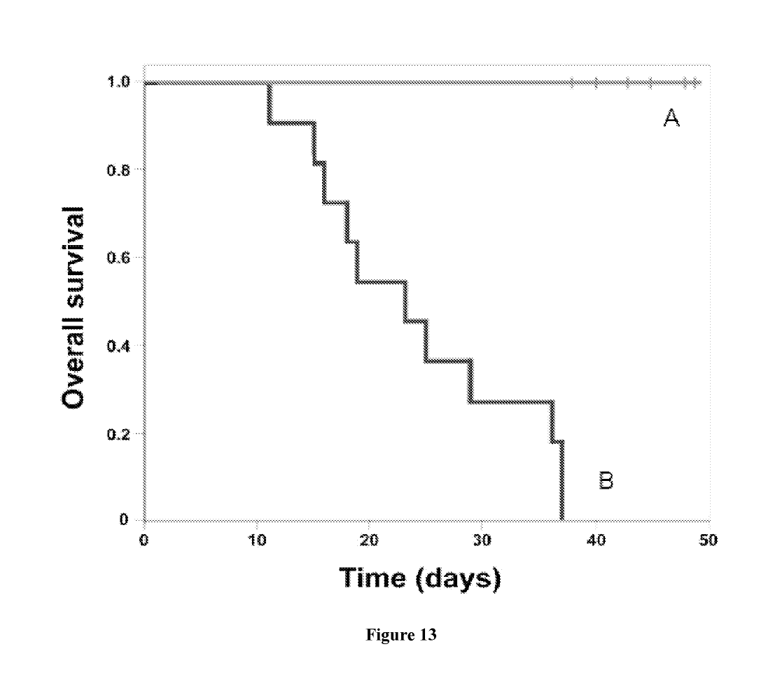

The results of the in vivo experiment depicted in FIG. 12 predicted that Shh antagonists would have a positive effect on a mouse model of cancer. Intracranial medulloblastoma model (conditional Patchedl null) mice used in this experiment develop medulloblastoma with an early onset and 100% penetrance. Accordingly, mice were injected daily with 20 mg/kg of either A) vehicle plus IPI-926 (n=12); or B) vehicle only (n=11). The mice were monitored for 50 days for survival, and the results depicted in FIG. 13. The experiment showed that mice given IPI-926 drug had significantly increased survival compared to the control mice. The results of this experiment demonstrate that Shh antagonism does effect cancer progression in vivo, as predicted by the in vivo experiment of FIG. 12, but not by the in vitro experiment of FIG. 11.

### Example 3

### Spatially-Restricted Delivery of Nucleic Acids

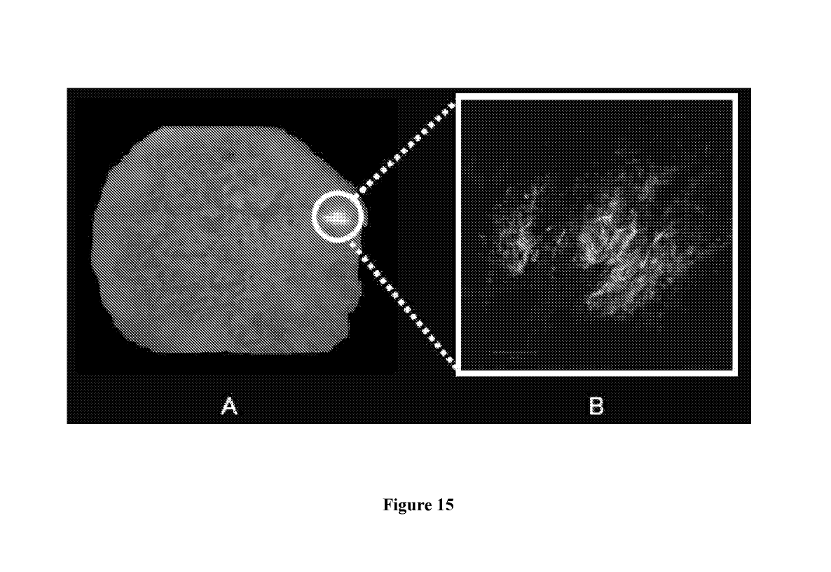

Spatially-restricted delivery of nucleic acid molecules was tested using the present method. A HT29 colon tumor xenograft was injected with lentivirus bearing a promoter driving GFP expression. FIG. 15 illustrates fluorescent microscopy of a whole tumor slice following spatially-restricted injection of GFP-expressing lentivirus. Panel A shows that GFP expression was localized to the region of injection. Panel B shows magnification of the virus infusion zone.

The method was then applied for spatially restricted RNA interference (RNAi). A small hairpin RNAi (shRNA) construct within a lentivirus was locally delivered to a mouse tumor. The shRNA was directed against KIF11, an essential gene for tumor cell mitosis. A control construct with no knockdown ability was also used. As an additional control, GFP virus alone was injected. These three constructs were injected at three different locations within the tumor, and localized effects were observed. In all three cases, the apoptosis reporter near-infrared tagged annexin 5 (Visen) was co-injected together with the constructs. Following injection, the tumor was excised, sectioned, and visualized by fluorescent microscopy to observe the apoptosis reporter.

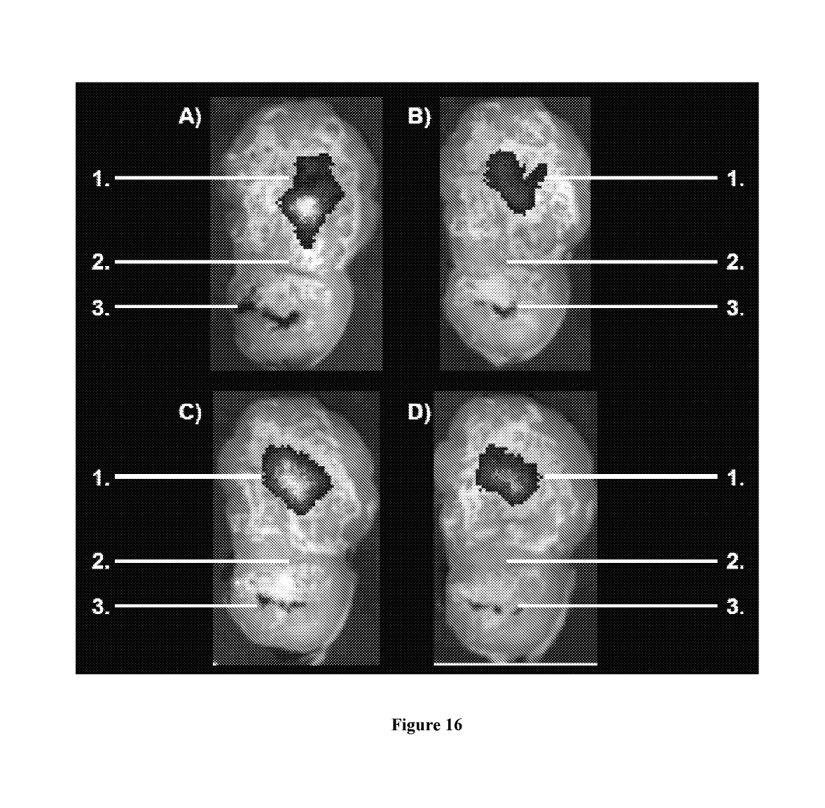

FIG. 16 depicts the results of this analysis, showing 1. KIF11 shRNA with an apoptosis reporter; 2. Control virus with an apoptosis reporter; and 3. GFP virus with an apoptosis reporter. Scans were taken at four tumor depths: 500 microns; 1,000 microns; 1,500 microns; and 2,000 microns. Only the region that received KIF11 shRNA shows a positive readout for apoptosis, indicating that tumor cell killing correlated with knockdown of KIF11, and was spatially restricted to the region that received the KIF11 shRNA construct.

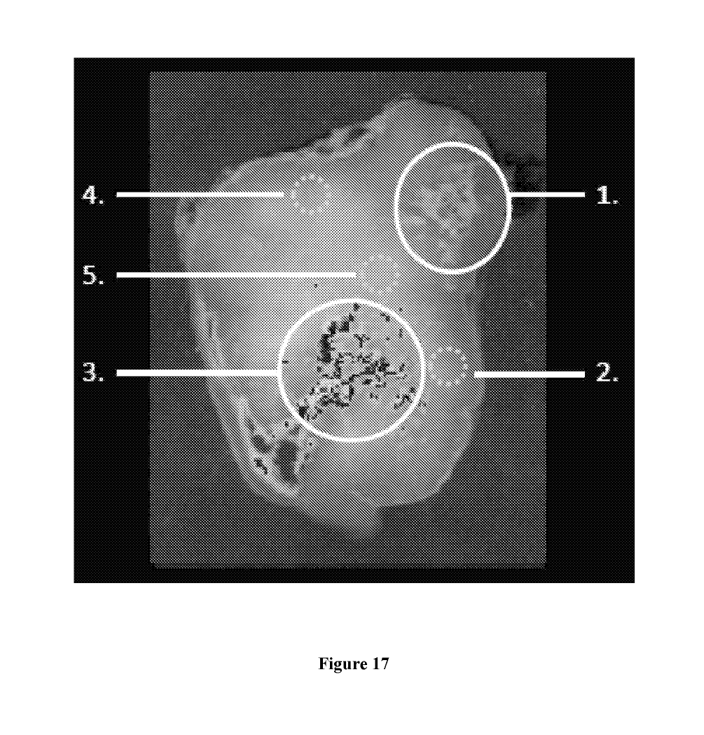

Spatially restricted tumor cell killing is also shown in FIG. 17. A mouse tumor was injected with 1. KIF11 shRNA; 2. Control; 3. KIF11 shRNA and an apoptosis dye (near infrared-tagged annexin 5); 4. Control; and 5. Control and the apoptosis dye. The tumor was excised, sectioned, and visualized by fluorescent microscopy. The results show that spatially restricted delivery of KIF11 shRNA, but not control, results in a signal observable using an apoptosis dye. No background signal was observed when KIF11 or control constructs were administered without the apoptosis dye.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A drug-delivery device for constrained solid delivery of one or more fluid agents to a tissue, comprising:
i) one or more needles each configured to receive a porous tube; and
ii) one or more porous tubes each configured to contain at least one fluid agent.

2. The drug-delivery device of claim 1, wherein at least one of said one or more porous tubes is biocompatible, permeable, and/or scissile.

3. The drug-delivery device of claim 1, wherein at least one of said one or more porous tubes comprises polysulfone, polyamine, polyamide, polycarbonate, polycarbamate, polyurethane, polyester, polyether, polyolefin, polyaromatic, polylactic acid, a cross-linked polymer, or a combination or co-polymer of any of the foregoing.

4. The drug-delivery device of claim 1, wherein at least one of said one or more porous tubes comprises polysulfone.

5. The drug-delivery device of claim 1, further comprising an actuator configured to push said one or more porous tubes from said one or more needles upon activation.

6. The drug-delivery device of claim 5, wherein said actuator is a plunger.

7. The drug-delivery device of claim 1, wherein each of said one or more needles is not permeable to said one or more fluid agents.

8. The drug-delivery device of claim 1, further comprising two or more reservoirs each in communication with a respective one of said one or more needles.

9. The drug-delivery device of claim 1, further comprising ten or more reservoirs each in communication with a respective one of said one or more needles.

10. The drug-delivery device of claim 1, further comprising one hundred or more reservoirs each in communication with a respective one of said one or more needles.

11. The drug-delivery device of claim 1, comprising two or more porous tubes.

12. The drug-delivery device of claim 1, comprising five or more porous tubes.

13. The drug-delivery device of claim 1, comprising ten or more porous tubes.

14. The drug-delivery device of claim 1, comprising one hundred or more porous tubes.

15. The drug-delivery device of claim 11-14, wherein none of the porous tubes comprises the same fluid agent as the agent in any other tubes.

16. The drug-delivery device of claim 1, wherein at least one of said one or more porous tubes comprises two or more different fluid agents.

17. The drug-delivery device of claim 1, wherein at least two of the porous tubes comprise a same fluid agent.

18. The drug-delivery device of claim 17, wherein the concentrations of the same fluid agent in different porous tubes are different.

19. The drug-delivery device of claim 1, wherein said one or more fluid agents are capable of diffusing through the pores at a diffusion rate when the porous tube is embedded in a tissue.

20. The drug-delivery device of claim 19, wherein the diffusion rates are controlled by the pore size.

21. The drug-delivery device of claim 20, wherein the pore size of porous tubes is a range between about 1 nm and about 5 micrometers.

22. The drug-delivery device of claim 1, wherein said one or more fluid agents comprise an anti-cancer agent, an anti-inflammatory agent, an anti-infective agent, a regenerative agent, a relaxing agent, an apoptosis-inhibiting agent, an apoptosis-inducing agent, an anti-coagulatory agent, a dermatological agent, a growth-stimulating agent, a vasodilating agent, a vasorestricting agent, an analgesic agent, or an anti-allergic agents.

23. The drug-delivery device of claim 1, wherein said one or more fluid agents comprise an anti-cancer agent.

24. The drug-delivery device of claim 1, wherein at least one of said one or more porous tubes comprises at least one indicator particle.

25. The drug-delivery device of claim 24, wherein said at least one indicator particle is selected from the group consisting of a metallic particle, a fluorescent dye, a quantum dot, a quantum barcode, a radiographic contrast agent, and a magnetic resonance imaging contrast agent.

26. The drug-delivery device of claim 24, wherein said at least one indicator particle is a fluorescent dye.

27. The drug-delivery device of claim 1, wherein said tissue is an animal tissue.

28. The drug-delivery device of claim 1, wherein said tissue is a human tissue.

29. The drug-delivery device of claim 1, wherein said tissue is a tumor.

30. The drug-delivery device of claim 1, wherein at least one of said one or more porous tubes is hollow.

31. The drug-delivery device of claim 1, wherein at least one of said one or more porous tubes uniformly comprises porous material.

32. The drug-delivery device of claim 1, comprising two or more needles.

33. The drug-delivery device of claim 1, comprising five or more needles.

34. The drug-delivery device of claim 1, comprising ten or more needles.

35. The drug-delivery device of claim 1, comprising one hundred or more needles.

36. The drug-delivery device of claim 32-35, wherein none of the needles comprises the same fluid agent as the agent in any other needles.

37. The drug-delivery device of claim 32-35, wherein at least one of the needles comprises two or more porous tubes.

38. The drug-delivery device of claim 32-35, wherein at least one of the needles comprises two or more fluid agents.

39. The drug-delivery device of claim 32-35, wherein at least two of the needles comprise a same fluid agent.

40. The drug-delivery device of claim 32-35, wherein the concentrations of the same fluid agent in different needles are different.

41. A method for spatially restricted solid delivery of one or more fluid agents to a tissue of an organism, comprising the steps of:
i) inserting one or more porous tubes into a tissue using one or more needles; and
ii) delivering the content of said one or more porous tubes to the tissue at least partially by diffusion through pores of said one or more porous tubes.

42. The method of claim 41, wherein the content of porous tube is delivered solely by diffusion.

43. The method of claim 41, wherein said inserting is performed with an actuator.

44. The method of claim 41, wherein said actuator is a plunger

45. The method of claim 41, further comprising loading said one or more porous tubes with said one or more fluid agents.

46. The method of claim 45, wherein said loading occurs by passive diffusion or osmosis.

47. The method of claim 45, wherein said loading occurs by capillary force.

48. The method of claim 45, wherein said loading occurs by contacting one end of each of said porous tubes to a fluid reservoir, and applying negative pressure to the opposite end of each of said porous tubes.

49. The method of claim 41, wherein two or more porous tubes are inserted.

50. The method of claim 41, wherein ten or more porous tubes are inserted.

51. The method of claim 41, wherein one hundred or more porous tubes are inserted.

52. The method of claim 49-51, wherein none of the porous tubes comprises the same fluid agent as the agent in any other tubes.

53. The method of claim 49-51, wherein the porous tubes are inserted along parallel axes within said tissue.

54. The method of claim 41, wherein at least one of said one or more porous tubes comprises two or more fluid agents.

55. The method of claim 41, wherein each of said one or more needles is not permeable to said one or more fluid agents.

56. The drug-delivery device of claim 49-51, wherein at least two of the porous tubes comprise a same fluid agent.

57. The method of claim 41, wherein at least one of said one or more porous tubes comprises polysulfone.

58. The method of claim 41, wherein the average pore diameter of pores of said one or more porous tubes is a range between about 1 nm and about 5 micrometers.

59. The method of claim 41, wherein said one or more fluid agents comprise an anti-cancer agent, an anti-inflammatory agent, an anti-infective agent, a regenerative agent, a relaxing agent, an apoptosis-inhibiting agent, an apoptosis-inducing agent, an anti-coagulatory agent, a dermatological agents, a growth-stimulating agent, an vasodilating agent, a vasorestricting agent, an analgesic agent, or an anti-allergic agent.

60. The method of claim 41, wherein said one or more fluid agents comprise an anti-cancer agent.

61. The method of claim 41, wherein said one or more fluid agents are delivered at or below systematically detectable concentration.

62. The method of claim 41, wherein said tissue comprises a tumor.

63. The method of claim 62, wherein said tumor comprises at least one cancer cell selected from the group consisting of a leukemia cell, a pancreatic cancer cell, a prostate cancer cell, a breast cancer cell, a colon cancer cell, a lung cancer cell, a brain cancer cell, a glioma cancer cell, a melanoma cell, a renal cancer cell, and an ovarian cancer cell.

64. The method of claim 41, wherein the porous tube remains in the tissue after the insertion.

65. The method of claim 41, wherein the delivery duration spans a selected period of time.

66. The method of claim 65, wherein said selected period of time is at least one minute.

67. The method of claim 65, wherein said selected period of time is a range between about 1 minute and about 96 hours.

68. The method of claim 65, wherein said selected period of time is a range between about one week and about six months.

69. The method of claim 41, further comprising evaluating the effects of said one or more fluid agents on said tissue.

70. The method of claim 69, wherein the evaluation comprises excising at least one portion of the tissue after introducing said one or more fluid agents.

71. The method of claim 70, wherein said excising occurs at a selected period of time after introducing said one or more fluid agents.

72. The method of claim 71, wherein the selected period of time is a range between about 1 minute and about 96 hours.

73. The method of claim 71, wherein the selected period of time is a period exceeding one week.

74. The method of claim 71, wherein the selected period of time is a range between about one week and about six months.

75. The method of claim 69, wherein the evaluation comprises pre-excising at least one portion of the tissue after introducing said one or more fluid agents.

76. The method of claim 69, wherein the evaluation comprises imaging the tissue.

77. The method of claim 76, wherein said imaging comprises radiographic imaging, magnetic resonance imaging, positron emission tomogoraphy, and biophotonic imaging.

78. The method of claim 76, wherein said imaging occurs before, during, or after introduction of said fluid agents.

79. The method of claim 69, wherein the evaluation comprises detecting an altered physiological state.

80. The method of claim 69, wherein the evaluation comprises determining and comparing the effects of at least two of the fluid agents on adjacent positions within the region of said tissue.

81. The method of claim 69, wherein the evaluation comprises determining the effects of at least two of the fluid agents on a same position within the region of said tissue.

82. The method of claim 69, wherein the evaluation comprises determining the effects of at least one of the fluid agents along the axis of insertion within the region of said tissue.

83. A method of rating a candidate agent for development into a therapeutic agent, comprising the steps of:
i) inserting one or more porous tubes containing one or more candidate agents into a tissue using one or more needles;
ii) delivering the content of said one or more porous tubes into the tissue at least partially by diffusion through pores of said one or more porous tubes; and
iii) evaluating the effects of said one or more candidate agents on the tissue.

84. The method of claim 83, further comprising one of (i) selecting at least one of said agents based on said evaluation; (ii) deselecting at least one of said agents based on said evaluation; and (iii) prioritizing at least two of said agents based on said evaluating.

85. The method of claim 83, wherein said one or more candidate agents are delivered at or below systematically detectable concentration.

86. The method of claim 83, wherein said tissue is a tumor.

87. The method of claim 83, wherein said one or more candidate agents comprise at least one position marker.

88. The method of claim 83, wherein said one or more candidate agents comprise at least one anti-cancer agent.

89. The method of claim 83, wherein the evaluation comprises excising at least one portion of the tissue after introducing said one or more fluid agents.

90. The method of claim 83, wherein the evaluation comprises imaging the tissue.

91. The method of claim 83, wherein said inserting is performed with a needle array device.
